(19) United States
(12) Patent Application Publication
(10) Pub. No.: US 2018/0028116 A1
(43) Pub. Date: Feb. 1, 2018

(54) PROPRIOCEPTIVE ENHANCEMENT DEVICE AND METHOD OF USE

(71) Applicant: Richard Fulton, Grand Junction, CO (US)

(72) Inventor: Richard Fulton, Grand Junction, CO (US)

(21) Appl. No.: 15/660,809

(22) Filed: Jul. 26, 2017

Related U.S. Application Data
(60) Provisional application No. 62/366,891, filed on Jul. 26, 2016.
(60) Provisional application No. 62/443,985, filed on Jan. 9, 2017.

(51) Int. Cl. A61B 5/00; A61F 5/01; A61F 5/32

(52) U.S. Cl. CPC A61B 5/6807; A61F 5/32; A61F 5/0111; A63B 22/0025

(57) ABSTRACT

A proprioceptive enhancement device, including methods of use, is described. The proprioceptive enhancement device includes a sock-like garment, with or without a shoe or other footwear, which mechanically and electrically stimulates cutaneous, subcutaneous, and deep tissue mechanoreceptors and proprioceptors to provide feedback to the user. The feedback causes the user to adjust weight distribution during athletic and non-athletic activities for optimal performance and increased overall health. The device includes additional configurations for electrically grounding the wearer to the earth, which may provide additional health benefits. The disclosure includes a method of using the proprioceptive enhancement device to treat wounds of the foot, including but not limited to venous stasis ulcers, arterial insufficient ulcers, traumatic wounds, and the like.

### CROSS REFERENCE TO RELATED APPLICATION

This application claims priority from U.S. Patent Application No. 62/366,891, filed Jul. 26, 2016 and entitled “Device and Method of Enhancing Postural Stability,” and additionally claims priority from U.S. Patent Application No. 62/443,985, filed Jan. 9, 2016 and entitled “Devices and Methods for Enhanced Electrical Conductivity and Postural Stability,”, the disclosures of which are hereby incorporated entirely herein by reference.

### BACKGROUND OF THE INVENTION

### Technical Field

The present invention relates generally to devices and methods for enhancing proprioception and increasing postural stability. Specifically, the present invention relates to a proprioceptive enhancement device and method of use.

### State of the Art

Balance and gait disturbances are the leading cause of accidental death and emergency room visits amongst older people. This public health issue creates enormous financial burden on the health care system reaching billions of dollars as well as untold human suffering. The problem will worsen as the population ages.

The extent of this problem is tremendous as 30% of those over 65 years of age and half of those over 80 years of age fall at least once per year. Greater than 90% of hip fractures result from falls, and 25% of those who fracture a hip die within 6 months. Even in survivors, a hip fracture results in a significant decrease in life expectancy.

While most falls do not result in hip fracture, the psychological effect and fear of falling results in a decline in the overall quality of life as a result of avoidance of physical and social activities. In fact, a large part of the mental energy of the aged is devoted to how to maintain balance and avoid falling, strategies for avoiding situations in which falling may occur, and constant worry that one may fall.

Postural instability and impaired balance in older individuals is a result in the age-related deterioration of several different systems that contribute to balance and proprioception. There is a complex interaction of systems, which involve primarily the visual, somatosensory system, and the vestibular system as well as the nervous receptors and spinal tracts. Visual changes with age include diminished visual acuity and depth perception amongst others. Proprioception, which is the ability to determine where the joints and limbs are in space, is diminished causing swaying and an increased reliance on vision to maintain balance. The transmission of impulses from the nervous receptors through the spinal cord and to the brain is also diminished. Vestibular weakness also occurs which may cause at least temporary disorientation. Other factors, which are more common with advanced age and may cause stability and balance problems, include decreased muscular strength, fear of falling, arthritis pain, poor judgment, coexisting medical conditions, and the drugs used to treat those conditions as well as the insulating effect of the foot from the earth by modern footwear amongst others.

Postural stability also affects many athletes as they age, and at a much younger age than affecting balance and gait disturbances of advanced age. The diminished athletic performance is a complex process, but it is known that proprioception abilities decline earlier than the loss of muscular strength and other factors important in athletic endeavors. Essentially, proprioception is the initial function to decline as one goes from early twenties to later twenties and beyond, and the decline in proprioception is integral to the decline in overall athletic ability, affecting muscular strength, coordination, and skills specific to that activity. Hence, since all sports require precise postural stability to achieve optimal results, improving postural stability may result in improved athletic performance. Essentially all active sports performances may be enhanced by providing a device and method to enhance balance.

Even in childhood, adolescence and early adulthood, postural stability is important in most every athletic endeavor from field and court sports, to bowling, to golf, to even archery. “It all begins in the feet” is a popular saying for many varied sports. Hence, increased proprioception methods have a potential to assist the athlete in developing and maintaining proper footwork and balance in mastering a particular athletic endeavor, whatever that endeavor may be. Providing a means to enhance the user's sensitivity to the weight distribution on the feet will contribute to better footwork and better overall balance and provide a platform for a better coordinated athletic move or motion.

Grounding, or “earthing,” is a term that describes efforts to achieve health benefits from walking outside either barefoot or with conductive footwear such as leather or performing indoor activities (working, sleeping, etc.) while connected to conductive systems that transfer the Earth's electrons from the ground via a ground wire or other conductive means into the body. Reported health benefits include stabilization of the immune system, diminished inflammation, normalized blood pressure, diminished cortisol levels, improved mood, diminished delayed onset muscle soreness after strenuous exercise, and protection against dementia amongst many other health benefits. Many of the grounding devices currently available act by grounding the individual with a sheet to sleep upon, pads, straps, or adhesive electrodes. While these means may help with restoring our natural neutral electric potential, humans historically have connected with the ground mostly through their feet while awake sixteen hours a day. They slept on nonconductive cellulose in the form of dried leaves, straw, and other nonconductive plant material to cushion themselves from the earth. Hence, being grounded during sleep may have some benefits, but it does not mimic the evolution of humans being primarily grounded while awake and primarily through the feet when awake. We evolved by being grounded primarily through channels which originate in the soles of our feet. This normal, natural method of grounding a human from a positive electric potential to the neutral electric potential of the earth and ridding one of the unnatural positive voltage that accumulates from various causes (including electromagnetic frequencies) during waking hours through the feet has been limited severely in the past 50 years with the conversion of footwear from a conductive leather to nonconductive synthetic products.

Leather is somewhat conductive and humans have used animal skins for footwear for eons. Especially with a bit of moisture from the environment or just from perspiration on the soles of the feet or both, footwear of leather and other animal products provides enough electrical conductivity to adequate ground the wearer. However, since leather does not provide complete dryness, becomes wet, is expensive, is difficult to configure a traction tread amongst other drawbacks, the vast majority of footwear utilizes other non-conductive man-made materials at least for the sole. This essentially insulates the user from the earth and the many readily encountered conductive substances covering the earth. Moreover, most humans spend the majority of their life indoors. Most homes and buildings are constructed with non-conductive materials that further insulate humans from the neutral electrical charge of the earth.

There is a clear need for novel devices which ground the user with improved means that are convenient and easy to use, unobtrusive, and may be utilized with the user's existing or chosen footwear to improve the user's health overall and improve their balance and proprioception particularly. Attractive yet novel devices will be described that successfully ground the user by means that may be used with the user's choice of footwear.

What is needed, therefore, is a device and method of use to create directed mechanical and electrical stimulation as feedback to a user of the device to optimize weight distribution for a variety of athletic and non-athletic activities, to increase opportunities for grounding the user electrically to the earth regularly throughout the day, and to improve overall health.

### DISCLOSURE OF THE INVENTION

The present invention relates generally to devices and methods for enhancing proprioception and increasing postural stability. Specifically, the present invention relates to embodiments of a proprioceptive enhancement device and methods of use.

Disclosed is a proprioceptive enhancement device comprising a foot garment having a cutaneous electrode and a pressure transducer; and a current generator electrically coupled to the cutaneous electrode, wherein the current generator delivers a current to the cutaneous electrode in response to a signal generated by the pressure transducer.

In some embodiments, the proprioceptive enhancement device further comprises a microprocessor communicatively coupled to the pressure transducer and the current generator; and an algorithm resident on microprocessor, wherein the algorithm directs the microprocessor to signal the current generator to deliver the current to the cutaneous electrode in response to the signal generated by the pressure transducer.

Disclosed is a proprioceptive enhancement device comprising a foot garment having an array, wherein the array comprises a plurality of stimulation members coupled to the foot garment; a conductive member coupled to the foot garment; and a shoe having a grounding element, wherein the grounding element is electrically coupled to the conductive member.

In some embodiments, the stimulation members are detachably coupled to the foot garment. In some embodiments, the plurality of stimulation members comprise a cutaneous electrode. In some embodiments, the plurality of stimulation members comprise a protrusion. In some embodiments, the plurality of stimulation members comprise a cutaneous electrode and a protrusion. In some embodiments, a position of the array on the foot garment is adjustable. In some embodiments, a shape of the array is adjustable. In some embodiments, the array is positioned on the foot garment corresponding with a position of an acupuncture meridian on a foot of a user. In some embodiments, the acupuncture meridian is a Kidney Meridian. In some embodiments, the array is positioned proximate to an ankle joint. In some embodiments, the ground element is biased against a ground surface by the actuator.

In some embodiments, the proprioceptive enhancement device further comprises a shoe support member coupled to the ground element and the connector.

Disclosed is a method of treating a foot wound comprising steps donning a proprioceptive enhancement device having a stimulation member onto a foot; stimulating a skin surface of the foot contacting the stimulation member in response to bearing weight of a user on a region of the foot; and causing the user to change weight bearing on a region of the foot.

In some embodiments, the stimulating step comprises generating a signal from a pressure transducer coupled to the proprioceptive enhancement device in response to bearing weight of a user on a region of the foot; and activating a current source to energize a cutaneous electrode coupled to the proprioceptive enhancement device.

In some embodiments, the method further comprises a step electrically grounding the foot to the earth by a ground element electrically coupled to the foot by the stimulation member.

The foregoing and other features and advantages of the present invention will be apparent to those of ordinary skill in the art from the following more particular description of the invention and its embodiments, and as illustrated in the accompanying drawing figures.

### BRIEF DESCRIPTION OF THE DRAWINGS

FIG. 1 is a plantar view of a right foot;

FIG. 2 is a medial view of a proprioceptive enhancement device worn on a right foot;

FIG. 3 is a dorsal view of a proprioceptive enhancement device;

FIG. 4 is a plantar view of a proprioceptive enhancement device;

FIG. 5a is a medial view of an proprioceptive enhancement device;

FIG. 5b is a plantar view of an alternative embodiment of a proprioceptive enhancement device;

FIG. 6a is a plantar view of an inner surface of a left-foot proprioceptive enhancement device;

FIG. 6b is a plantar view of an inner surface of a right-foot proprioceptive enhancement device;

FIG. 7 is a plantar view of an additional embodiment of a proprioceptive enhancement device;

FIG. 8 is a schematic representation of a proprioceptive enhancement device with an electrical stimulation feedback mechanism;

FIG. 9 is a medial perspective view of a conductive member of a proprioceptive enhancement device;

FIGS. 10a-b are perspective views of a conductive member of a proprioceptive enhancement device mounted on a nonconductive shoe;

FIG. 11 is a side view of a conductive member of a proprioceptive enhancement device;

FIGS. 12a-b are perspective views of a shoe support member of a proprioceptive enhancement device mounted on a shoe;

FIG. 12c is a plantar view of a shoe support member of a proprioceptive enhancement device;

FIG. 12d is a cross-sectional view of an insole of a proprioceptive enhancement device;

FIG. 13 is a perspective view of an alternative embodiment of a conductive member of a proprioceptive enhancement device mounted on a shoe;

FIG. 14 is a front-view of an alternative embodiment of a conductive member of a proprioceptive enhancement device;

FIG. 15 is a top-view of an insole of a proprioceptive enhancement device;

FIG. 16a-e are top views of several alternative embodiments of a conductive insole of a proprioceptive enhancement device;

FIG. 17 is a medial cross-sectional view of a sole mounted on a shoe of a proprioceptive enhancement device;

FIG. 18 is a top-view of an insole of a proprioceptive enhancement device;

FIG. 19 is a side view of an alternative embodiment of a conductive sock of a proprioceptive enhancement device;

FIG. 20 is a side view of an additional embodiment of a conductive sock of a proprioceptive enhancement device; and

FIG. 21 is a flowchart showing a method 200 of treating a foot wound utilizing a proprioceptive enhancement device.

### DETAILED DESCRIPTION OF EMBODIMENTS OF THE INVENTION

As discussed above, this disclosure relates generally to devices and methods for enhancing proprioception and increasing postural stability. Specifically, the present invention relates to embodiments of a proprioceptive enhancement device and methods of use.

Humans have been connected to the earth naturally for 500,000 years and have used leather and animal skins on their feet. Our body has evolved to be grounded through the feet during that time. A combination of the recent change in the construction of most footwear which effectively insulates a person from the ground and the dramatic increase in electromagnetic fields has caused the human body to be limited in its ability to function optimally. Measured voltages of up to 7 volts have been recorded during the development of the disclosed invention in individuals in different environments while wearing nonconductive footwear whereas the voltage approximated zero a short time after they were connected to a ground wire. These abnormal, unnatural inherent positive voltages that occur in the ungrounded state alter the body's release of hormones, neurotransmitters, electrical impulses, cellular permeability, mitochondrial activity, and many other functions. The body may cope and remain mostly healthy, but since the systems are not operating at peak capacity and peak performance in a finely tuned manner, there is a moderate chance an electrochemical reaction will incite a cascade of events that results in inflammation, which is the root cause of most all illnesses and maladies and responsible for all the conditions mentioned previously. Anxiety is a major product of being ungrounded. Anxiety is present during waking hours primarily and providing grounding solutions the grounding dilemma while the individual is awake are described below.

Hence, one goal of this invention is to provide novel means and methods of electromechanically conductively connecting the individual with the neutral electrical potential of the earth in novel ways through the feet. Additionally, configurations of devices and methods of use of devices that ground an individual generally including all of the body or focally ground particular areas of the anatomy will be discussed. These may be very important in restoring a natural state of electric potential especially in this day of the unavoidable bombardment of electromagnetic fields from the electronic devices and appliances that surround us and with which we interact for hours and hours each day.

One unreported health benefit is improved proprioception and balance. The instant inventor has experienced improved proprioception and balance from both physical stimulating means and direct electrical connectivity with the ground. In a prototype device which combines both, the limited initial results have been rather dramatic.

Research into postural stability and balance issues has identified many issues involved in balance and strategies for addressing these issues. There appears to be no universal remedy, but a strategy of addressing the multiple different body systems deficits, i.e. muscular, visual, proprioceptive, etc., and some effort directed toward rehabilitation of these deficits is needed. Research has neglected the contribution of grounding to postural stability, balance and proprioception

Hence, another aim of the current invention to provide a device and method to assist in improving postural stability and balance by overcoming the recognized age-related loss of cutaneous and mechanoreceptor sensation that is important to balance control either by providing stimulating means that encourage balance or discourage improper weight distribution which may be combined with grounding means to accentuate or restore the electrical connectivity with the earth. Another aim is to provide a device and method of improving the sensitivity of minor changes in positions that improve balance, footwork and reactions in various sports and activities with at least one of either stimulating means, discouraging means, or conductive means. Another aim is to provide a device and method that assists in postural stability by evoking mechanoreceptors in the ankle joint by at least one of either stimulating means, discouraging means, or conductive means as well.

Other uses of grounding described subsequently will detail improvement in age-related skin changes and devices and methods to address that issue. Even another use is to treat skin ulcerations from any of various causes, including diabetes and peripheral arterial or venous disease amongst others.

In summary, the scientific literature contains studies which point to the importance of cutaneous receptors in the skin of the sole of the foot, mechanoreceptors in the deeper tissues and joints of the foot and ankle, the transmission of these stimuli to the spinal cord and brain and how these components interact to aid in postural stability and balance. Peripheral receptors and their stimuli are just one part of the complex multi-system interactions that occur to provide postural stability and balance. Optimal balance may result from efforts to improve muscular strength, visual acuity, physical activity, as well as specific physical therapy and other means. Embodiments of the disclosed invention are directed toward enhancing the importance of specific cutaneous receptors and mechanoreceptors in the foot and ankle by mechanical means, electromechanical means, and both, to assist in improving balance, proprioception, and postural stability, and preventing falls in the aging population, providing a sense of confidence in ambulating, provide enhanced athletic abilities in those in which diminished coordination has occurred, and even improving athletic performance in a younger set of the population.

In the prior art, Maki, et al, in U.S. Pat. No. 6,237,256, describe a shoe insert with means of mechanically stimulating cutaneous pressure sensation from the perimeter of the plantar surface of the foot to enhance balance. This device may help, but it is limited in several ways. Mainly, it limits the stimulation means to the periphery of the feet. The narrow ridge about the perimeter of the insert may provide pressure, but the pressure will vary from subject to subject as several variables interfere with the universal usage of this device. Socks that are usually worn between the sole of the foot and the device will dampen or diminish the stimulatory effects of the narrow ridge somewhat depending on the thickness of the socks. An insert will not intimately contact the skin of the feet so that the means of stimulation, i.e., the small ridge like structures or other peripherally positioned structures, do not efficiently transmit the stimulus to the mechanoreceptors within the tissues and joints. Also, feet are shaped different from person to person, and one configuration for all comers may not provide a similar stimulation from person to person. Moreover, mechanoreceptors and other proprioceptors in the foot and ankle joint play a large role in postural stability. Most of the weight on the foot when standing is concentrated in general three areas: under the heel and under the head of the first and fifth metatarsals. Providing additional means of stimulation over these critical areas which are major contact points of the foot within a shoe may further enhance the feedback necessary for balance improvement. A subject needs to perceive that their weight and pressure are actually being placed on these three focal central areas in addition to having sensory input from the periphery of the foot. Additionally, providing means of stimulation about the ankle joint will provide additional stability. All of these means are lacking from the Maki patent. Moreover, Maki utilizes stimulating means along the complete side of the foot, both medially and laterally and around the heel pad. With this much stimulation, “noise” is created and the brain is unable to distinguish which sensation is the correct one for proper stability or position and which is not.

Focusing the stimulation on the more important areas of beneath the heel and under or slightly posterior to the heads of the first and fifth metatarsals would overcome this fault of the Maki patent. Moreover, the Maki disclosure emphasizes stimulating areas in which the weight should not be present in an attempt to alert the patient not to sway, and Maki makes no effort to stimulate the areas that the patient or user should attempt to stimulate for proper balance and position. This is of critical importance as the current disclosure may comprise means for stimulating the foot in those areas that are most important for balance and weight position to teach the patient or user the correct position to assume rather than just warn them of the incorrect position. The current devices may also comprise means of stimulating areas the areas in which weight should not be present and may comprise “encouraging” and “discouraging” means of stimulating both areas to encourage proper weight distribution and position and discourage improper weight distribution and position. Moreover, and potentially as or more important than the stimulating and discouraging means, the current invention may comprise a grounding means to restore on enhance electrical connectivity with the earth which may likely enhance the actions of the stimulating and discouraging means as well as provide a stand-alone means of enhancing balance and proprioception. Hence, the current invention offers a much more complete solution than the prior art in that it may teach the user not only what not to do, but what to do instead, and varying degrees of what not to do and what to do within the same device. Incorporating these and other features into a device which provides intimate contact while conforming to the foot with either multiple peripheral areas or focal central areas of stimulation will improve the effectiveness of the device. Incorporating a grounding connectivity feature will provide a much more complete solution than any prior art device.

In the prior art, there are several devices aimed at diminishing electrostatic discharge in a workplace environment for workplace safety that include U.S. Pat. Nos. 4,249,226, 7,609,503, 6,707,659, 5,576,924, and 5,004,425 amongst others. Other prior art devices are described in U.S. Pat. Nos. 6,682,779, 7,212,392, and 7,724,491 amongst others are directed to personal grounding and removing positive charges from the body to achieve health benefits while sleeping. The former group of prior art devices may be functional in a work environment, but have severe limitations when applied to normal daily activities and/or sporting events. The grounded footwear in this group tend to be sturdy, robust but non-stylish footwear. The add-on grounding straps that are used with standard footwear are viewed as clumsy, bulky, inconvenient to secure and obtrusive in any location other than the workplace. Both the current grounded footwear and the current add-on grounding straps and devices would not be chosen to be worn outside of a workplace that demanded electrostatic discharge protection because of their inconvenience and appearance. Similarly, the latter group is directed to means of grounding the user primarily during sedentary activities including sleeping and sitting or include tethered connections that prevent grounding during ambulation or sporting activities.

The distinction of the current invention from prior art devices is disclosed herein. Many different uses of the technology of the current invention are anticipated which may include physical therapy after injury, surgery, stroke, periods of inactivity or convalescence, to initiate and foster proper ambulation with proper weight distribution, as well as the above-mentioned examples amongst others. This use may be in the form of self-directed use, medical directed use, instructor directed use, caregiver directed use, and the like. For example, in the post-operative period, it is important for all patients to ambulate as soon as possible after surgery. This is difficult at times because of the pain from the surgical wound. Having stimulating means beneath the feet to focus upon, will not only instruct the patient in the correct weight position and transfer, but may cause the patient to be distracted from the discomfort of the surgical wound by focusing on the stimulating means, thereby accelerating the ambulation process. Grounding may not only increase the sensitivity to the stimulating means, and also diminish the inflammation responsible for the pain of the surgical wound, allowing earlier and more vigorous ambulation.

The current invention solves the problems in the prior art by providing stimuli producing means and/or electrically conductive means in at least, but not limited to, one or more of the following locations: about the periphery of the foot, in the three major foot contact areas, about the K-1 acupuncture meridian, about the great toe, and about the ankle. The current invention may incorporate the stimuli means with or without electrically conductive means within a sock or foot/ankle sleeve to fully conform to the foot, to make intimate and direct contact with the foot, and provide a stimulating means or electrically conductive means to specific areas of the foot and/or about the ankle, all of which are not present in the prior art. Other embodiments may utilize conductive means in one or more of shoes, inner soles, socks, foot sleeves, liners, connecting leads and the like to accomplish the same.

In much of the prior art, a grounding cord is used to conductively connect the body to the ground. This is inconvenient at times, a burdensome tether that limits freedom of movement. This may not be the case with grounded pads while sleeping, but even sitting or standing on a grounded object without conductive clothing or footwear will insulate the individual and prevent grounding. Hence, it would be beneficial for there to be a means of grounding the individual without the use of a bothersome grounding cord, and that is another goal of this invention. The whole purpose of grounding an individual is to neutralize the electrical charge within the body with the electrical charge of the earth. Even if the body is insulated from the earth, nature provides means of neutralizing the charge differences. Human skin and hair normally possess a negative electrical charge as does animal skins and furs. This is a result of exposure to the sun and is nature's method of providing a secondary means of neutralizing one's body even without direct or indirect contact with the ground. Most individuals at this point in history spend the vast majority of their time indoors, in cars, and out of the sunlight which can provide neutralizing powers. Under the inescapable bombardment of electromagnetic fields and insulation from the earth, the body's internal electrical charge becomes positive and is inhibited from being neutralized by the ground or sunlight. The dilemma becomes how to wirelessly ground an individual to the earth absent a grounding wire with a wireless grounding means, essentially. Most internal floors are insulated from the earth by non-conductive building materials. For indoor use, the user may employ the above embodiments along with one or more commercially available grounded pads or mats on the floor on which to place the feet. These mats or pads, which may be connected to the ground of an electrical outlet, may be placed under a desk so that the user may be grounded while working or engaged with a computer, at the foot of a chair so that the user may be grounded while relaxing or watching television, or at any location that the user desires so that by wearing one of the embodiments above and placing the grounded foot and apparatus on the mat or pad, the user will be effectively grounded. Hence, this arrangement overcomes the use of a tether which may restrict the mobility of the user.

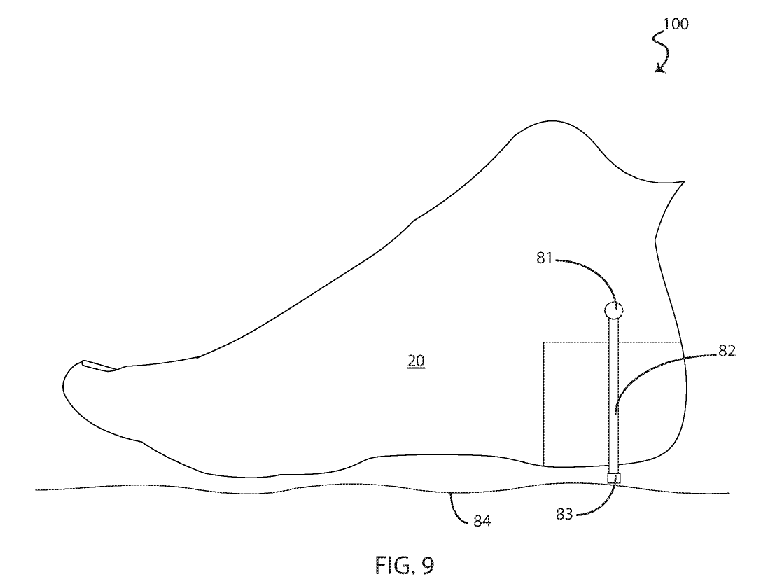

Another component may consist of an electrically conductive shoe inner sole which may serve to conductively connect the conductive sock to the conductive shoe or shoe connectivity means, or may just be in contact with the user's foot in the absence of socks. Hence, several components of a conductivity “system” may be employed to connect the skin to the ground while wearing both socks and shoes. The inner sole may be made conductive by utilizing conductive materials or by conductive filaments placed within standard inner sole materials. The conductive inner sole may be constructed to make conductive contact with the conductive connectivity means 91 of FIG. 9 described above. While certain components of specific embodiments are shown with other components, any of the components described herein may be utilized with or combined with any of the other components to facilitate conductivity from the ground to the user. As well, there are multiple conductive inner soles and shoes in the prior art, including U.S. Pat. Nos. 7,055,266, 5,233,769, 4,150,418, 4,642,912, 4,926,570, and 6,003,247. The embodiments of the current inventions may be utilized with prior art or other devices to complete the “system” of connectivity.

Additionally, a sock-like structure or inner sole may comprise means which ground the user to the earth and concentrate the grounding on the sole of the feet at an area known as the acupuncture meridian of “Kidney 1” or “K-1,” located centrally in the ball of the foot just posterior to the metatarsal heads. This is the only acupuncture meridian on the sole of the foot and is a major energy vortex that has the ability to affect mind, body and spirit. This point and meridian may be activated not only by acupuncture, but also by acupressure (focal pressure), exercise, topical pastes or gels, and the like. The Kidney 1 meridian has been referred to as the “Bubbling Spring,” as it is the entry portal for the earth's energy to travel up and then throughout the body. The conductive sock-like structure or inner sole structure may comprise conductive materials woven into the fabric of the structure or added to the structure after or during the weaving process. There may be a relative increased concentration of the conductive material within the woven fabric with methods known in the industry or added to the woven fabric near the site that the sock-like structure or inner sole structure contacts the Kidney 1 meridian of the foot. Moreover, the woven or added conductive materials may radiate out from or inward towards the Kidney 1 meridian contact area in any one of a plurality of patterns even including a spiral pattern amongst others. Hence, the grounding efforts may be concentrated at the Kidney 1 meridian which will facilitate the effects of grounding.

Commercially available Earthing Shoes may provide a conductive member, which may be comprised of a conductive metal, a conductive polymer, or other conductive substances, and which pierces the sole and may be attached to conductive straps in a thong like sandal configuration or just pieces the sole of the shoe. This conductive member may be from less than a millimeter in diameter to several centimeters in diameter. Others may place a conductive member through the shoe sole to contact the earth with the exterior aspect and contact the skin of the sole of the foot with the interior aspect of that member in an insert like configuration. Both configurations are designed to be worn without socks which can be problematic in the winter or at any time a sock is needed or preferred for comfort, cushioning, warmth, or protection amongst other reasons. Both configurations also may create an aperture in the sole which may not prevent the egress of water or other substances to the interior of the shoe. Neither of these drawbacks may be important for casual wear in warm climates, but may be undesirable to be used in a daily work day, sporting activities, on a rainy day, normal walking, with business attire, and the like as the user's socks may insulate the wearer from the grounding mechanism provided by the grounded or Earthing Shoe essentially nullifying the purpose and effect of the grounded shoe. Hence, one aspect of the present invention is directed to providing an earthing or grounding system that provides conductive connectivity with the person and the earth while wearing standard non-grounded shoes as well as enhancing the conductivity of the grounded or earthing shoes described above.

While the above described footwear, including socks, shoes, inner soles, grounding apparatuses and other components, may well ground the user and return the user's inherent electrical potential to near zero and that of the earth, a more natural way would be to mimic the grounding methods that our species has been using for eons. Animal skin shoes have been discovered from the mid to late Paleolithic Era, 40,000-100,000 years ago, and it appears that our species has been wearing them in one form or another until about 50 years ago when the switch to nonconductive manmade materials occurred. Woven socks have been discovered from the very early Egyptian area, although it is likely that most of the populace may not have used socks as we know them until more recently, and the materials and loose weave, used in all but the most modern times, would certainly allow conductivity in the presence of some perspiration.

The soles of the feet are one of the most nerve-rich parts of the body. The three most highly innervated parts of the body are the hands, the face (particularly the lips) and the feet. Why the feet? The feet (when bare) are the only part of the body that is in constant contact with the environment. With over 100,000 nerve endings per foot, tactile feedback and other sensory input from the soles of the feet provide a wealth of information to the brain about the ground upon which one treads. Whether one is walking or running, that information is used to make adjustments (within milliseconds) to the gait, the goal always being to reduce impact forces on the joints and body. These sensory endings contribute to maintenance of balance, muscle coordination, and essentially any activity done while erect. Of course, this sensory information is also used to warn you of dangerous terrain or injurious objects. Unfortunately, most footwear creates a ‘shoe-induced neuropathy’ because the thick outer sole and cushioned innersole eliminates sensory feedback including lack of electrical conductivity.

Humans have evolved so that the largest concentration of sweat glands in the body is on the soles, or plantar surface, of the feet. Why was this? It is plausible that one major reason was and is to maintain and promote electrical conductivity with the earth via the perspiration on the soles of the feet. The electrolytes within perspiration enhance the conductivity of the animal skins and leather to keep the user grounded when ambulating. Essentially, the individual evolved from a barefoot state with the entire area of the soles of the feet being grounded to a barefoot state within an animal skin covering. to enhance balance, proprioception, tactile senses related to enhanced muscular coordination and many other activities. Recently we have evolved to being in a barefoot state but within an insulated shoe. One reason that the foot perspires so much in footwear of manmade nonconductive materials is that the body is seeking connectivity with the earth, and, since this connectivity is lacking, the sweat glands are stimulated to secrete more sweat in an effort to make a connection with the earth. Hence, to mimic the grounding of and from the soles of the feet that has occurred over the eons, footwear that transmits the grounding to the entire plantar surface of the foot may be beneficial. This may include a sock with conductive materials limited to the sole portion of the sock that is contacted by the plantar surface of the foot. Also, an inner sole which is completely conductive over its entire surface is contemplated. The entire shoe sole may be made of leather or other manmade conductive material, rather than utilizing a grounding connector outside the shoe or a single or a few conductive member(s) which may pierce the nonconductive sole of the shoe. Since many conductive materials do not provide the durability of nonconductive materials and may be more expensive, another configuration may provide more uniform connectivity to the earth and may comprise multiple thin strips, wires or the like of a conductive material in the nonconductive sole. These may vary in number from several to hundreds, but preferably from 20-100. They may course throughout the sole, but in a preferred embodiment the thin strips, wires, or the like may extend from the bottom of the sole to the interior of the sole in a more or less vertical axis to the sole of the shoe or sole of the user or both. In this instance, if the foot rests on the interior of the sole, then “micro grounding” of the sole of the foot would occur, first with the heel and subsequently along the plantar surface to the balls of the feet and then to the toes in sequence when ambulating. The weight bearing surfaces of the plantar aspect of the foot would experience micro grounding in those areas in which weight distribution is most desired for balance. In the case of inner soles, the inner sole may have a similar construction with conductive means oriented in a more or less vertical axis to either the sole of the foot or the sole of the footwear or both. When combined with a sock comprised of a sole constructed of conductive materials only in the sole, then the mimicking of our ancestors will be complete and contact with the earth will be more natural and more in line with our evolutionary history. Hence, improvements in balance, proprioception, muscular coordination, sensation perception, agility, and all of the processes related to ambulating, standing, climbing, running, and the like may be expected Enhanced sports performance may also be expected. If the soles of the feet are connected in this manner, then perspiration or sweating of the feet will likely be diminished as there will be fewer stimuli to produce perspiration to achieve connectivity with the earth. The method of diminishing foot perspiration by grounding the soles of the feet by either direct contact with a conductive shoe sole or with a conductive means and a sock with the conductive material only in the sole of the feet as described above or elsewhere in this disclosure is another object of this invention.

While many medical conditions may benefit from the various methods and devices described herein, one condition that is particularly difficult to treat is ulcers of the skin of the feet that may be secondary to arterial or venous insufficiency, diabetes, and neuropathy. Venous ulcers are generally caused by venous insufficiency and/or venous obstruction which prevent the normal flow of venous blood from the foot area towards the heart as a result of incompetent venous valves and/or obstruction of the veins resulting in increased pressure in the venous system of the feet causing the skin to ulcerate. In arterial insufficiency, the blood supply to the foot is compromised. In diabetic and other neuropathies, the sensitivity of the skin to stimuli is diminished causing skin erosion. An inflammatory reaction occurs which further worsens the situation and prevents or at least partially limits therapeutic efforts directed to healing these skin ulcerations. Providing a means and method of accelerating foot ulceration healing is another object of this invention. The method may involve using any one of the embodiments previously described or placing conductive means that may be connected to said embodiments on the skin near the venous ulcerations. The subject or patient would then wear the grounding footwear apparatus. Since most chronic venous ulcer patients don't ambulate much and spend the majority of their time indoors, and since indoor surfaces are not usually conductive, the subject or patient may utilize a grounded floor pad at the foot of a chair while watching TV or relaxing, at the foot of a desk chair while computing or working, under their feet when elevating the feet to overcome venous stasis, in an area of frequent standing (stove, sink, etc.), and in those locations they may frequent amongst others. The grounding apparatus may be incorporated into support hose or other devices that treat the condition and then further connected to the foot grounding means and the floor grounding pad for example. The patient may also utilize grounding while sleeping described elsewhere or herein to augment the grounding described herein done while awake.

The foregoing disclosure describes a proprioceptive enhancement device, including stimulation members and grounding members, which overcome many of these noted deficiencies of the prior art. The disclosure includes methods of use, and methods for using a proprioceptive enhancement device to treat wounds of the foot.

Standard anatomic reference points are used throughout this disclosure to describe position of structures. Such anatomic reference terms describing positional relationships in the foregoing disclosure may include, but are not limited to, plantar, dorsal, anterior, posterior, cutaneous, subcutaneous, proximal, distal, medial, lateral, and the like. Wherein an anatomic term is used to describe the position of a non-anatomic structure, such as a structure comprised by an embodiment of the invention, anatomic terms describe the relationship of the structure to the anatomy of a user of the invention. All of the anatomic terms used in the foregoing disclosure mean standard anatomic relations or reference points known to those in the healthcare and medical device arts.

It is to be understood that commonly accepted meanings be attributed to additional terms are used throughout this disclosure. Some non-limiting examples of such meanings included. For example, as used herein, “shoe” means any item of footwear having a generally rigid sole in contact with the ground or a standing surface, including, but not limited to a shoe, a sandal, a slipper, and the like. “Foot garment” means any item worn on the foot, including shoes, socks, slippers, footies, and the like. “Ground surface” means the surface of the earth, or any standing surface; i.e., floor, road, etc. electrically grounded to the earth by any means. “Wound” means a discontinuity in the epithelium of the skin, without limitation, including a venous stasis ulcer, and arterial insufficiency ulcer, a traumatic injury, a surgical incision or surgical wound, and the like. “Earthing” means establishing electrical conductivity between a body of a person and the earth, for any length of time, wherein the overall electrical potential difference between the body of the person and the earth is approximately zero.

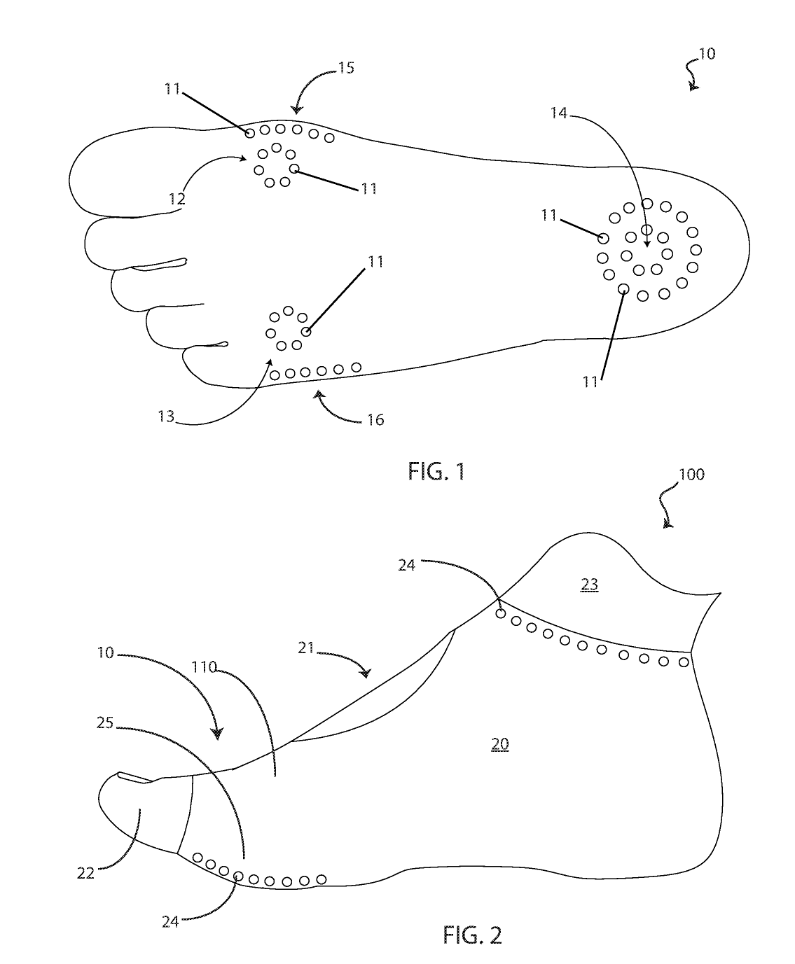

FIG. 1 is a plantar view of a right foot. FIG. 1 shows a foot 10 having a weight-bearing region 11. Weight-bearing regions 11 are areas of skin on the plantar (sole-of-the-foot) skin surface of a foot. The small circles in FIG. 1 representing weight-bearing regions 11 are arranged in the shape of an array representing the general size and shape of weight-bearing regions 11, a plurality of which exist on each foot. Weight-bearing regions 11 shown in FIG. 1 include three critical areas: 1) first weight bearing region 12 is located just posterior to the head of the first metatarsal 2) second weight-bearing region 13 is located just posterior to the head of the fifth metatarsal; and third weight-bearing region 14 is located beneath the heel pad. Proprioceptors and mechanoreceptors (not shown) located in the skin and subcutaneous regions of weight bearing regions 11 generate neural signals which provide a triangulating effect of sensation. These signals are integrated with other sensory input by neural processing centers in the brain and spinal cord to provide balance. Weight bearing regions 11 of lesser importance include the medial edges of the foot medial to the head of the first metatarsal 15 and lateral to the head of the fifth metatarsal 16. Proprioceptive receptors in the ankle (not shown) also contribute to the position of foot 11 when ambulating, running, or changing direction. These concentrations of mechanoreceptors and proprioceptors located proximate to weight bearing regions 11 contribute more than other similar receptors in other areas of the foot, hence it is important to target these areas for stimulation when attempting to provide sensory feedback regarding weight position and postural stability. These and all stimulation members described herein, whether encouraging or discouraging stimulation members, may be combined with electrical conductivity or grounding means.

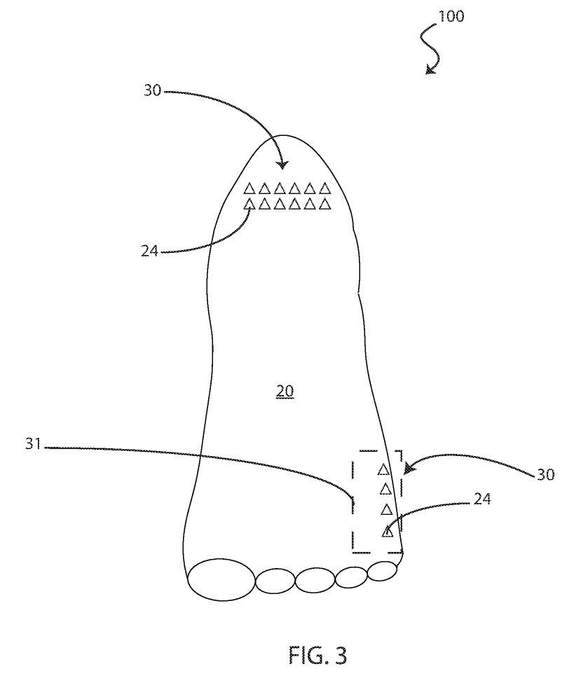

FIG. 2 is a medial view of a proprioceptive enhancement device 100 worn on foot 10. Proprioceptive enhancement device 100 comprises a foot garment 20. FIG. 2 shows a medial perspective of a foot garment 20 formed as a foot-ankle sleeve to be worn by a user. Foot garment 20 may be formed from any possible number of fabrics commonly used to construct a foot garment, such as synthetic or natural-weave fibers, elastomeric compounds and weaves including Lycra, natural or synthetic rubbers, and the like. Some embodiments of foot garment 20 have an open area 21 over the dorsum of the foot. Foot garment 20, in some embodiments, covers a plurality of toes 22; in some embodiments, foot garment 20 at least partially covers toes 22. In some embodiments, foot garment 20 extends proximally to cover an ankle joint 23. Although FIG. 2 shows an outer surface 110 of foot garment 20, a plurality of stimulation members 24 are shown positioned and coupled to foot garment 20 along the medial aspect of the foot adjacent to the head of the first metatarsal and the first metatarsophalangeal joint 25, and proximate to ankle joint 23. Although FIG. 2 shows outer surface 110 of foot garment 20, stimulation members 24 protrude through to an inner surface 102 (not shown in FIG. 2; See FIG. 3). In some embodiments, a sock is worn over foot garment 20. Alternatively, in some embodiments, an athletic support tape may be applied by the user to cover an outer surface of foot garment 20, a sock may be placed over the tape, and a shoe may be worn over the sock.

In the embodiment shown by FIG. 2, and in some other embodiments, stimulation members 24 are positioned on an inner surface of foot garment 20 which contact the skin of foot 11 when foot garment 20 of device 100 is worn by a user, providing direct stimulation to cutaneous nerve receptors and substantial stimulation to the subcutaneous and deeper mechanoreceptors and proprioceptors upon weight bearing the user. Such stimulation is an effective means of providing stimulatory feedback to the body, including reflex neural loop pathways. Direct contact of stimulation members 24 with the skin of foot 10 eliminates the damping effect of a sock that may be worn within the shoe and is placed between stimulation members described by the prior art and the skin of the feet. Moreover, as demonstrated in FIG. 2 and in some embodiments, stimulation members 24 are placed about ankle joint 23. As the feet are the body's anchor to the ground and the ankle joint connects the foot to the rest of the body, the ankle joint is important, if not central, in the complex interaction of balance, footwork, athletic moves, human locomotion, weight distribution and transfer, amongst others, and not simply a convenient articulator at the end of the leg. The proprioceptors and mechanoreceptors within the ankle joint reflect the position of the foot relative to the lower leg. The cutaneous and subcutaneous receptors however “fine tune” sensations gathered from within the joint. Hence, some embodiments of proprioceptive enhancement device 100 comprise stimulation members 24 proximate to ankle joint 23, as shown by FIG. 2, to provide additional sensory feedback to a user wearing foot garment 20 of device 100.

Stimulation members 24 must stimulate cutaneous receptors, subcutaneous receptors, deep tissues, and joints without being uncomfortable to the user when weight bearing. Inadequate stimulation is not beneficial and too intense stimulation is uncomfortable or painful such that device 100 will not be used. In embodiments wherein stimulation members 24 are placed directly in contact with the skin of foot 11 adequate degrees of stimulation are provided while maintaining comfort, therefore, expanded choices of the shape and size of stimulation members 24, in some embodiments, is desirable for device 100 to be effective and comfortable to use. Stimulation members 24, in some embodiments, comprise discrete individual excrescences, expanded areas of surface inconsistencies, continuous ridges, and the like. In some embodiments, stimulation members comprise any one or more of several shapes including, generally, spherical, hemispherical, cylindrical, square, rectangular, triangular, pyramidal, toroidal, ellipsoidal, multi-faceted, circular, pin-like, or a combination thereof. Stimulation members may be comprised of any one of a number of materials including, but not limited to, plastics, polymers, metal, fabric, Velcro, metals and alloys including copper and other metals, other suitable materials, or a combination thereof.

FIG. 3 is a dorsal perspective view of a proprioceptive enhancement device. FIG. 3 shows inner surface 102 of foot garment 20 with foot garment 20 “turned inside out.” In the embodiment shown by FIG. 3, and in some other embodiments, stimulation members 24 comprise small triangular excrescences coupled onto a surface of or embedded within the substance forming foot garment 20 in to stimulate selected areas, grouped in an array 30. In some embodiments, array 30 comprising a plurality of stimulation members 24 is positioned on foot garment 20 to encourage the user to adopt a defined foot positon or weight distribution advantageous for a particular athletic move or motion. Alternatively, such as in the case of elderly balance instability, array 30 is positioned in a lateral location 31 to encourage the user not to place too much weight laterally on the foot. In some embodiments, array 30 is fixedly coupled to foot garment 20, which may comprise the sock, sleeve, shoe insole, or the like. In some embodiments, array 30 modifiable, wherein individual stimulating members 24 are detached from foot garment 20 and repositioned to create any variety of shapes and locations comprised by array 30. This arrangement of specific patterns of array 30, in some embodiments, is performed by the user/wearer of foot garment 20, a physical therapist, or other training or healthcare personnel, according to the desired application of proprioceptive enhancement device 100.

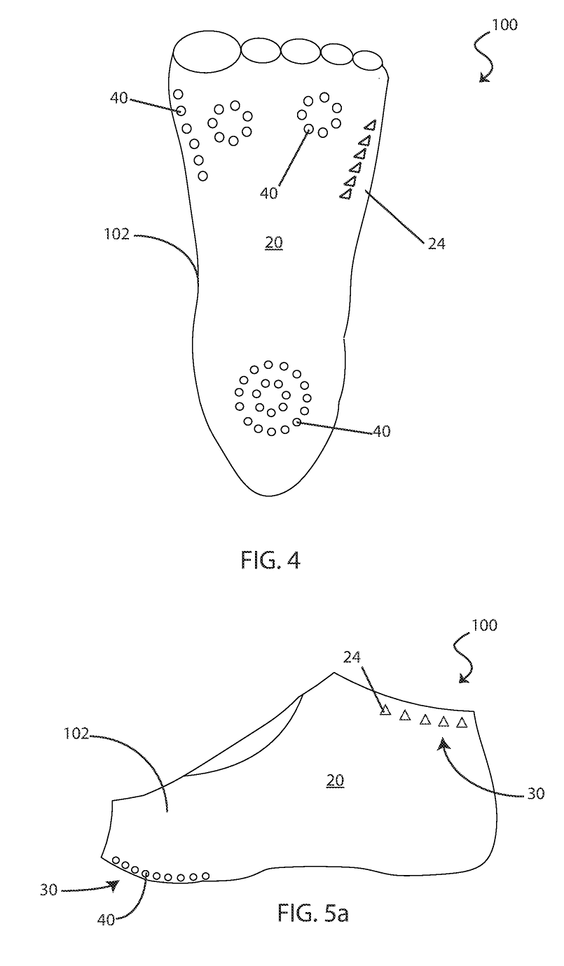

Some embodiments of foot garment 20 comprise a generic distribution of attachment couple for stimulating members 24 to form array 30. In other words, the attachment couple (not shown in FIG. 3, See FIG. 4) may provide for different patterns to be utilized by the user or other person, whereupon stimulating members 24 are coupled to certain of the attachment couple. For example, some embodiments of foot garment 20 may comprise a distribution of attachment couple that will accommodate different configurations of stimulation members into arrays 30, depending on the specific need of a specific user. For example, stimulation members 24 are positioned into array 30 comprising a first shape and located in a first position on foot garment 20 for tennis and a second shape located in a second position for golf Stimulation members 24 may be arranged in any variety of arrays 30, depending on the need of the proper weight-bearing locations and weight transfer dynamics for these and other specific activities and applications.

FIG. 4 is a plantar view of a proprioceptive enhancement device. FIG. 4 shows inner surface 102 of foot garment 20, wherein foot garment 20 is shown turned “inside out.” FIG. 4 shows an attachment couple 40, represented by a circle. A plurality of attachment couples 40 are arranged in a larger shape, wherein coupling of a plurality of stimulation members 24 (shown in FIG. 4 by triangles) to a plurality of attachment couples 40 form array(s) 30. In the embodiment shown by FIG. 4, and in some other embodiments, stimulating members 24 only occupy a minority of the attachment couples 40 that are positioned laterally on the forefoot are of foot garment 20. Such embodiments may be used when a coach or athletic instructor identifies a specific fault in weight distribution and transfer that required improvement; therefore, stimulation members 24 are positioned to form in a particular-shaped array 30 to address any one of a number of possible faults in a specific individual user of device 100.

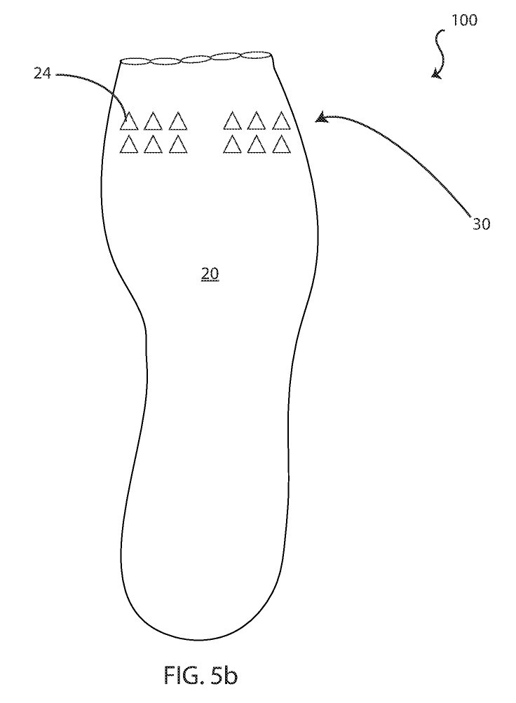

FIG. 5a is a medial view of a proprioceptive enhancement device. FIG. 5b is a plantar view of an alternative embodiment of a proprioceptive enhancement device. FIGS. 5a-b show examples two distributions of stimulation members r24 into arrays 30 positioned proximate to first weight-bearing region 12 and second weight-bearing region 13. Stimulation of receptors in first weight-bearing region 12 and second weight-bearing region 13 encourages a classic “athletic ready position,” wherein a user of device 100 centers weight over the forefoot and bends the ankles forward, allowing the user/athlete to transition forward, backward, or sideways during a sports activity, such as basketball, football, or similar court or field sports. An array 30 of stimulation members 24 placed anterior to the ankle 23 gives the user feedback that the ankles are in a partially flexed position, effecting knee and hip flexion. The utility of this particular location of array 30 proximate to ankle 23 (not shown in FIG. 5a-b) cannot be over emphasized. Additionally, arrays 30 of stimulating members 24 positioned proximate to first weight-bearing region 12 and second weight-bearing region 13 encourage the user to place more weight forward on the feet. The combination of arrays 30 encouraging forward weight positioning and ankle flexion is important at all experience levels of athletic activity, but particularly for a beginner striving to correctly learn fundamentals and ingrain proper techniques for a sport.

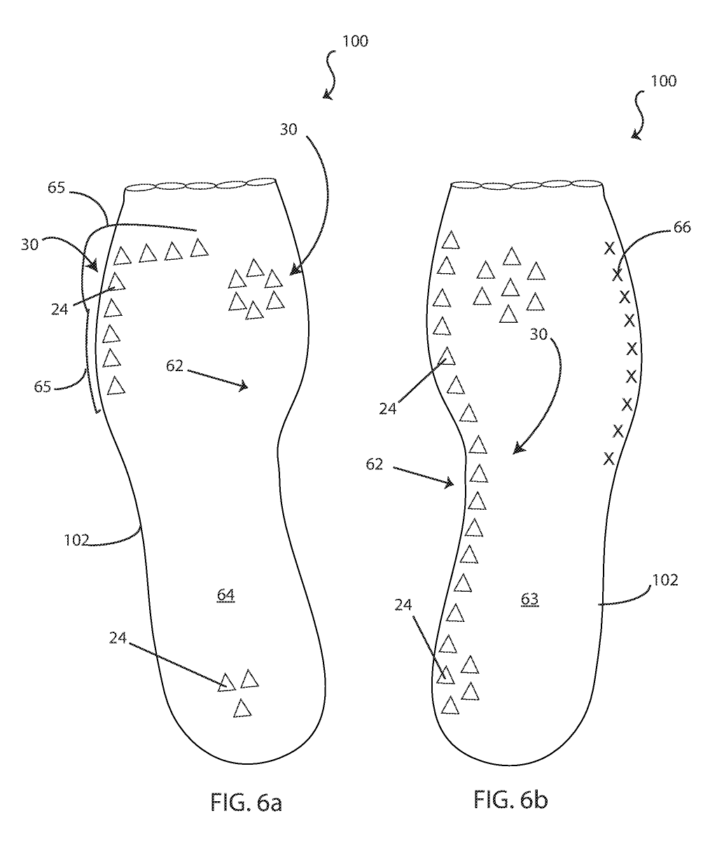

FIG. 6a is a plantar view of an inner surface of a proprioceptive enhancement device and FIG. 6b is a plantar view of an inner surface of a proprioceptive enhancement device. FIGS. 6a-b show a plantar aspect of inner surface 102 of two devices 100 which are turned “inside out-mirror images” for illustration purposes, to demonstrate an embodiment of device 100 bearing arrays 30 of stimulation members 24 to encourage proper weight distribution during a typical swing or stroke for a right-handed golfer. Stimulating members 24 are configured array 30 to encourage weight distribution along a medial edge 62 of a right foot garment 63, versus a left foot garment 64. Note that array 30 along medial edge 62 of right foot garment 63 comprises a greater number of individual stimulation members 24 than array 30 on the left foot 64. The number or concentration of stimulation members 24 in a particular area provides sensory feedback to the user to reposition weight. In this example, stimulating members 24 are concentrated beneath a first metatarsal head 60 and along medial edge 62 of right foot garment 63. This positioning encourages a user/golfer to place more weight where array 30 of stimulation members 24 is positioned on foot garment 20 in response to sensory feedback generated by stimulation members 24. In the example embodiment shown in FIGS. 6a-b, and in some other embodiments, arrays 30 of left foot garment 64 comprise fewer stimulation members 24, signaling the user that less weight is borne by the user's left foot, generally. Array 30 shaped in a curvilinear line 65 formed by stimulation members 24 on left foot garment 64 represent a desired progression of weight transfer during a golf-swing follow-through, wherein device 100 provides the golfer sensory feedback to optimally execute dynamic weight transfer necessary for a proper golf swing.

In some embodiments, array 30 of stimulation members 24 is positioned for static weight distribution, as in the athletic ready position described herein above, or for the progression of weight transfer onto the left foot also described. Stimulation members 24 may be positioned on foot garment 20 to encourage either for static weight bearing or progression/weight shifting, and may be formed into different shaped arrays comprising materials of different densities and surface textures, and so on. Likewise, if there are particular or optimal weight positions for a specific sport or activity, array 30 may be configured, in some embodiments, with a distribution of stimulation members 24 customized to that specific sport or activity.

In some embodiments, array 30 of stimulation members 24 is formed to encourage one particular weight distribution, and an alternative array 30 of stimulation members 24 is formed to discourage an alternative weight distribution pattern. Array 30 is shaped and positioned so the user may adjust distribution across different foot regions to either seek or avoid stimulation caused by stimulation members 24. Additionally, in some embodiments, stimulation member 24 intended to encourage a position may comprise a size, shape, resiliency, and texture to create a more-or-less pleasant sensation, wherein stimulation member 24 intended to discourage a position may comprise an alternative size, shape, resiliency, and texture to create an unpleasant sensation. A non-limiting example of stimulating member 24 formed to generate an unpleasant sensation upon weight bearing comprises a prickly or irritating surface texture upon weight bearing. Possible examples of such an irritating surface texture are provided by the hooks of a hook-and-loop closure system (i.e., Velcro), a tubular braid, pin like projections, a cylindrical tubular structure, frayed wire, or any essentially any irritating shape or substance. As shown in FIG. 6a-b, an unpleasant stimulation member 66 may be placed laterally on right foot garment 63 to discourage rolling of the right foot and an improper swaying weight transfer during a golf backswing for example.

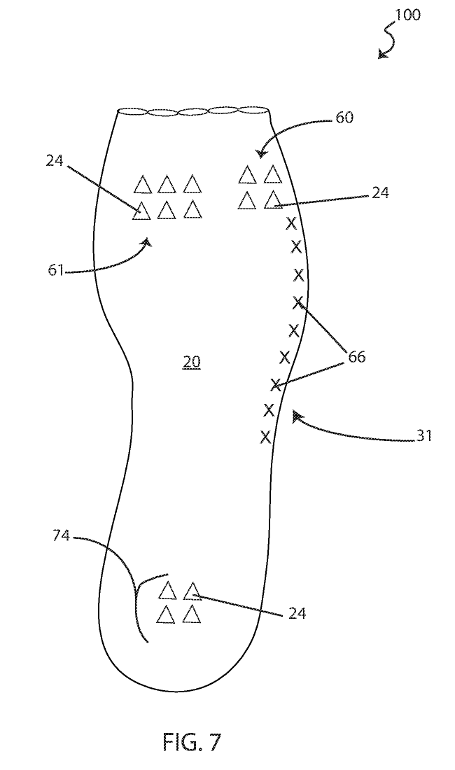

FIG. 7 is a plantar view of an additional embodiment of a proprioceptive enhancement device. In some embodiments, discouraging stimulation members 24 are positioned in foot garment 20 beneath the heel regions, or alternative areas, to discourage weight placement in these regions. Stimulation members 24 encouraging or discouraging weight bearing may be used independently or together in a single foot garment 30. FIG. 7 shows a use of encouraging stimulation members 24 beneath and slightly posterior to the metatarsal heads of the first metatarsal head 60 and fifth metatarsal head 61 metatarsals and beneath the heel region 74. This distribution of stimulation members 24 tends to encourage a user, such as an elderly user, to distribute weight over first metatarsal head 60, fifth metatarsal head 61 and heel pad 74 to increase postural stability. Unpleasant stimulating members 66 may be placed along the lateral sides 31 of the feet to discourage rolling of the feet and the subsequent loss of balance. Unpleasant stimulation members 66 may also be placed about the lateral aspects of the ankle (not shown in FIG. 7) to alert the user of improper position of the ankle to further prevent falling. The user may be instructed to seek out the sensation of the encouraging stimulation members 24 when walking and standing and to avoid the sensation of unpleasant stimulation members 66.

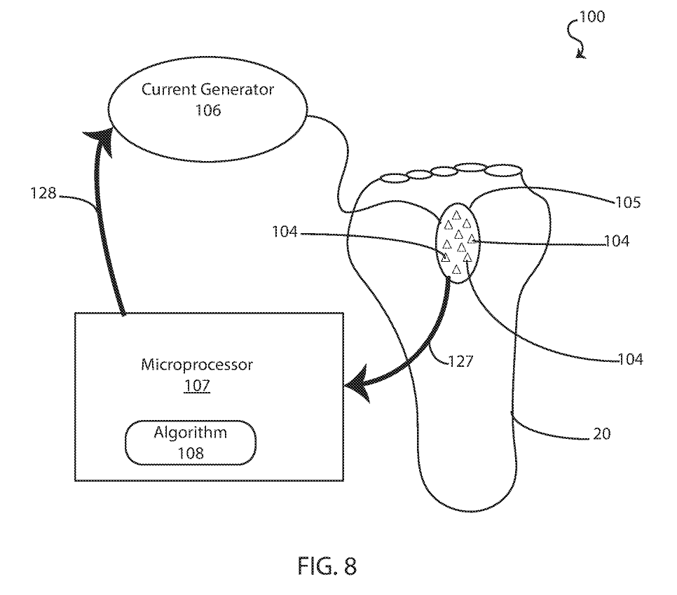

FIG. 8 is a schematic representation of a proprioceptive enhancement device with an electrical stimulation feedback mechanism. FIG. 8 shows a cutaneous electrode 104, a pressure transducer 105, and a current generator 105. FIG. 8 additionally shows a microprocessor 107 and a software application 108. In some embodiments, proprioceptive enhancement device comprises an electronic user weight-distribution feedback mechanism wherein pressure transducer 105 is coupled to weight bearing region 11 of foot garment 20. The weight-distribution feedback mechanism is controlled by software application 108 resident on microprocessor 107. Microprocessor 107 is communicatively coupled to pressure transducer 105 and also communicatively coupled to current generator 106. Under a condition wherein a user dons foot garment 20 of some embodiments of proprioceptive enhancement device 100 having the electrical stimulation feedback mechanism, and bears weight on weight bearing region 11, pressure transducer 105 responds to the weight-bearing and generates a first signal 127 directed to microprocessor 107. In response to first signal 127, microprocessor generates a second signal 128 directed to current generator 106 causing current generator 106 to energize a stimulation member 124.

Pressure transducer 104 is a device to electronically detect weight, transmitted as pressure at a specific location on the sole of the foot, such as a weight-bearing region 111, for example. Pressure transducer 104 is any of a multiplicity of suitable pressure transducers which are commercially available and widely known to those in the medical device and electronic arts. Current generator 104 comprises a battery and an electronic activation switch, in some embodiments, and is one of many widely available current generators suitable to applications discussed herein. Cutaneous electrode 104 is formed from a metal or other suitable electrically conductive material. In some embodiments, stimulation member 124 or unpleasant stimulation member 166 comprise cutaneous electrode 104, such that a stimulation member may create stimulation or unpleasant stimulation through tactile or electrical effect, or both simultaneously, according to the desired application and use of device 100.

Microprocessor 107, in some embodiments, is provided with device 100. In some embodiments, microprocessor 107 is a separate element, such as a mobile computing device, including a smartphone or a tablet computing device. In some embodiments, microprocessor 107 is electrically coupled to current generator 106, cutaneous electronic 106, or both current generator 1′06 and cutaneous electrode 105. In some embodiments microprocessor 107 is wirelessly communicatively coupled to current generator 106, cutaneous electronic 106, or both current generator 106 and cutaneous electrode 105. Wireless communicative coupling is accomplished using Bluetooth, a cellular network, a radiofrequency device, or the like, as are widely available and ubiquitous in many geographic areas.

Software application 108, in some embodiments, is any smartphone app, desktop computer application, or the like comprising software code programming an algorithm to receive and process first signal 127 from pressure transducer 105, including a plurality of pressure transducers 105, and interpret first signal 127 to determine an activation pattern for stimulation member(s) 124 or unpleasant stimulation member(s) 166.

In some embodiments of device 100, therefore, it is possible to measure a baseline weight distribution and then suggest an improved or optimal distribution of weight, through software application 108 resident on microprocessor 107, for the user of device 100 to achieve. In some embodiments, such baseline weight distribution is saved as data in a memory (not shown) comprised by a computing device, such as a desktop computer, a mobile computing device, and the like. A second software application 108 (not shown), in some embodiments, receives and processes the data to generate a suggested configuration of stimulation members 24 or unpleasant stimulation members 66 in array 30, and to generate a size of array 30 and a position underlying a region of the user's foot to encourage the calculated optimal weight distribution and discourage sub-optimal weight distributions and standing positions. These data, representing baseline and optimal positioning measurements, is electronically transmitted to a designer or manufacturer of device comprising array(s) 30 specific to the needs of a specific individual. A method, therefore, comprising at least one of assessing an individual's baseline condition, suggesting an improvement, and creating a device in which the configuration of stimulation members 24, unpleasant stimulation members 66, or both stimulation members 24 and unpleasant stimulation members 66 may encourage the improved position, discourage the incorrect position, or both simultaneously, and subsequent use by the individual to achieve the improvement or avoid the incorrect position in some embodiments. Further, the method may be accomplished by mechanical stimulation members, electronic stimulation members, or a combination of mechanical and electronic stimulation members. It should be understood that functions of arrays 30 of stimulation members 24 or unpleasant stimulation members 166 are incorporated into the electrical stimulation feedback mechanism described herein above.

Stimulation member 24, stimulation member 124, unpleasant stimulation member 66, and unpleasant stimulation member 144 are formed from any one of many different materials, including but not limited to fabric, plastics, fiber, metals, metal alloys, minerals, wood, plant material, chemical formulations, electrical impulse conducting/transmitting surfaces and materials and other substances. Further, stimulation member 24, stimulation member 124, unpleasant stimulation member 66, and unpleasant stimulation member 144 are either coupled electrically or wirelessly with current generator 106 or microprocessor 107. Further, the shape(s) of stimulation member 24, stimulation member 124, unpleasant stimulation member 66, and unpleasant stimulation member 144 may be at least partly cylindrical, spherical, rectangular, square, triangular, serpiginous, figure of eight, straight, pointed, rounded, “v” or “w” shaped, multiple, singular, repetitive, varied or mixed amongst others. The consistency may be firm, soft, hard, mixed, varied, and so on. Stimulation member 24, stimulation member 124, unpleasant stimulation member 66, and unpleasant stimulation member 144 coupled to foot garment 20 may be singular or may be grouped together in any one of a number of sized and shaped arrays 30 as discussed at length herein. The size of each stimulation member 24, stimulation member 124, unpleasant stimulation member 66, and unpleasant stimulation member 144 may vary depending on the member's shape and number, and the material forming the member, but varies generally within a range of 0.10 millimeters and 5 millimeters. In some other embodiments, stimulation member 24, stimulation member 124, unpleasant stimulation member 66, and unpleasant stimulation member 144 may be of an elongate shape and extend for over 40 cm in greatest length. A thickness of stimulation member 24, stimulation member 124, unpleasant stimulation member 66, and unpleasant stimulation member 144 may be important in imparting a sensation that is perceived but not uncomfortable to wear. The thickness of any stimulation member 24, stimulation member 124, unpleasant stimulation member 66, or unpleasant stimulation member 144 depends on the material utilized in formation, and the maximum width may range between about 0.01 millimeters and 2 millimeters.

Furthermore, any configuration or distribution of stimulation member 24, stimulation member 124, unpleasant stimulation member 66, and unpleasant stimulation member 144, may be combined, either within an array 30 or outside of an array 30, with any other configurations, size, shape, material or consistency even though that combination may not be explicitly stated or referred to herein. Any configuration or distribution of physical or mechanical stimulation members may be combined with other means that may be chemical, electrical, grounding, magnetic, or the like.

In some embodiments, proprioceptive enhancement device 100 comprises a grounding element 83 electrically coupled to a conductive member 82 and a cutaneous electrode 81 configured to facilitate electrically grounding the user of device 100 to the earth. In some embodiments, cutaneous electrode 81 is comprised by any or all of stimulation member 24, stimulation member 124, unpleasant stimulation member 66, and unpleasant stimulation member 144. Further, stimulation member 24, stimulation member 124, unpleasant stimulation member 66, and unpleasant stimulation member 144, as described herein, concomitantly apply pressure to the proprioceptors and mechanoreceptors of the feet, and may act as grounding and stimulating means, may act as only grounding means, or may act as only stimulating means. Stimulation member 24, stimulation member 124, unpleasant stimulation member 66, and unpleasant stimulation member 144, in some embodiments, are electrically coupled to grounding element 83 by conductive member 82, described in detail herein below.

FIG. 9 is a medial perspective view of a conductive member of a proprioceptive enhancement device. In the case of a sock or sock-like structure worn by the user over foot 10 inside a shoe or a shoe insole, grounding element 83, although incorporated into footwear, will not be effective unless electrical conductivity is established with the earth (i.e., electrical grounding) through coupling with a conductive aspect of the footwear and then from the shoe to the ground. A system is, therefore, needed that combines a conductive shoe or shoe component that electrically coupled with foot garment contacting skin of the user. FIG. 9 shows an embodiment of device 100 comprising conductive member 82 electrically coupled to grounding element 83 and cutaneous electrode 81. In the embodiment shown by FIG. 9, and in some other embodiments, cutaneous electrode 81 comprises foot garment 20 formed from conductive material. In some embodiments, conductive member 82 releasably coupled grounding element 83 to cutaneous electrode 81. The example grounding mechanism comprised by device 100 shown in FIG. 9 contacts a grounded surface 84 upon which the user is standing. Grounded surface 84 is the earth or an electrically conductive surface electrically coupled to the earth. Grounding element 83 is formed of any conductive material and may be flexible, inflexible, partially flexible, or it may combine any of these features. The point of contact between grounding element 83 with grounded surface 84 comprises an adequate surface area to provide adequate electrical grounding.

Alternatively, in some embodiments, grounding element 83 of device 100 as shown in FIG. 9 electrically contacts a plantar surface of the user's skin directly through the material forming a conventional sock, rather than a conductive sock, such as wherein grounding element 183 pierces material forming the sock to make conductive contact with the skin of the foot or ankle. A shoe (not shown) is normally worn over the sock.

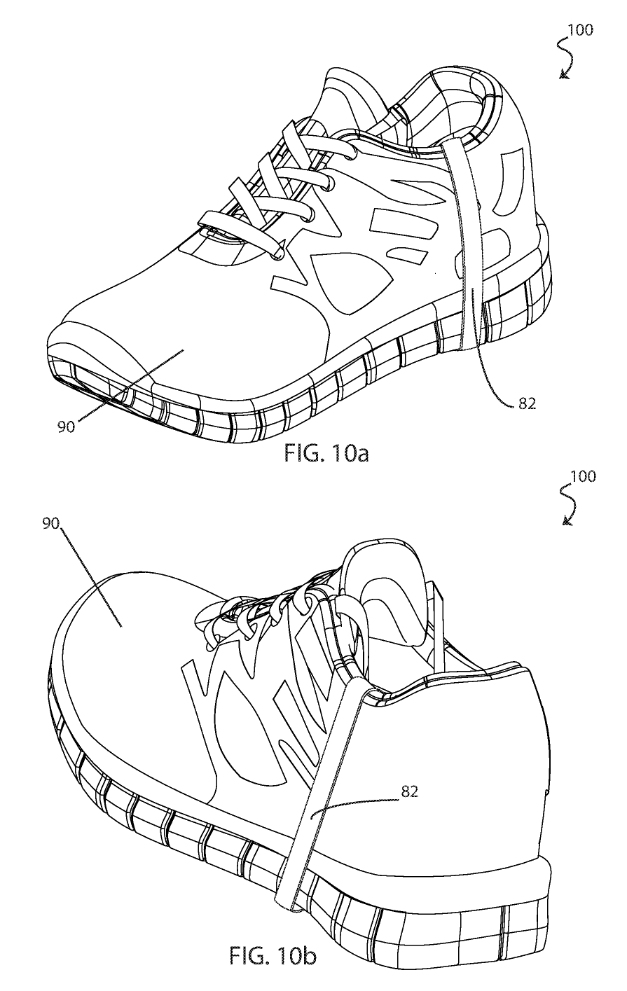

FIGS. 10a-b are perspective views of a conductive member of a proprioceptive enhancement device mounted on a nonconductive shoe. FIGS. 10a-b show an example of yet another embodiment of proprioceptive enhancement device 100 comprising grounding element 83. FIGS. 10a-b comprise conductive member 82 coupled to a shoe 90. Conductive member 82 may be coupled with essentially any standard non-conductive commercially available shoe 90 to couple cutaneous electrode 81 of garment 20 (not shown) to grounding element 83 (also not shown). In some embodiments, conductive member 82 is a U-shaped member formed to slidably couple over the edge of shoe 90. In some embodiments, conductive member 82 is releasably coupled to shoe 90 by friction. Additionally, cutaneous electrode 81 (not shown) may electrically coupled to conductive member 82 by contacting a surface of a conductive sock, a non-conductive sock rendered conductive by the user's perspiration, a conductive insole, or directly with a skin surface of the user. In some embodiments, conductive member 82 is preferably attached to the medial aspect of shoe 90 to be visually discreet while in use.

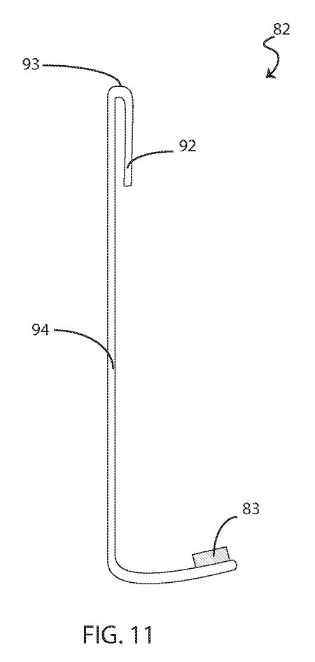

FIG. 11 is a side view of a conductive member of a proprioceptive enhancement device. FIG. 11 shows conductive member 82 having an internal arm 92, a bend 93 which fits over the edge of a shoe, and an external arm 94. The external arm 94 extends underneath the shoe where it terminates at grounding element 83. In some embodiments, grounding element 83 is shaped to fit within a groove or depression in the shoe sole, such as a groove or depression in the tread of the sole, to mitigate wear experienced when grounding element 83 forcefully contacts with the ground with each step which would necessitate frequent repair or replacement. Grounding element 183, in some embodiments, is positioned at the arch of the shoe, a position which receives substantially less force than a standing surface. In these and some other similar embodiments, grounding element 83 is relatively protected. To maintain contact with the standing surface, however, an actuator 96 (not shown) is operatively coupled to grounding element 83 wherein grounding element 83 is biased against a ground surface by actuator 96. Actuator 96 is any suitable actuator mechanism, including a compressed coil spring, a compressed flat spring, a resilient segment of conductive member 182, or a similar mechanical or electromechanical device that urges grounding element 83 out of the groove in the sole of the shoe to contact the ground or standing surface. In some embodiments, bend 93 is formed from a resilient material wherein internal arm 92 and external arm 94 are biased toward one another such that conductive member 82 grips the side of a shoe by friction, fixing grounding element 83 in position. A suitable means of coupling grounding element 83 or actuator 96 to the receiving feature of the shoe sole may comprise an adhesive, a barb, a screw, and the like to mechanically coupled conductive member 82 to the user's shoe. In some embodiments, grounding element 83 is magnetically coupled to a corresponding magnetic feature formed into the shoe sole. Alternatively, grounding element 83 comprises a conductive screw coupled directly to the shoe sole, in some embodiments.

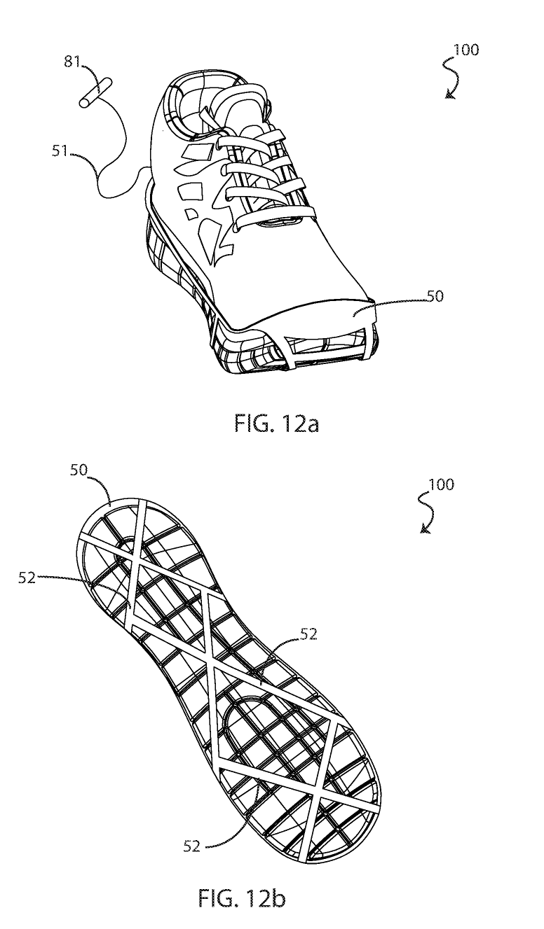

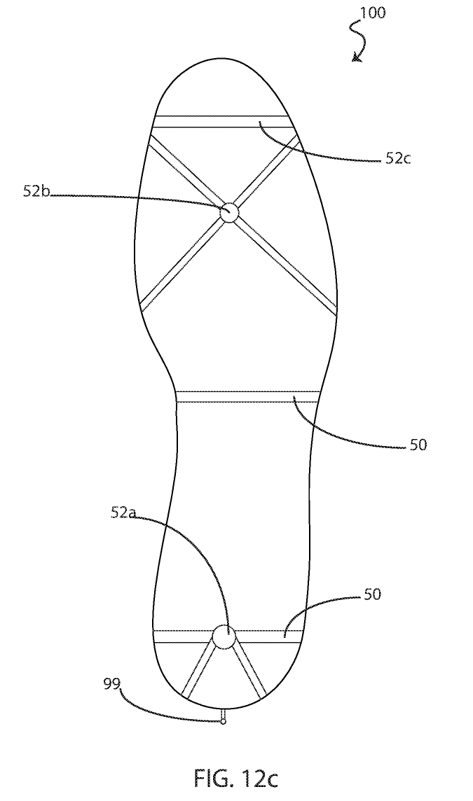

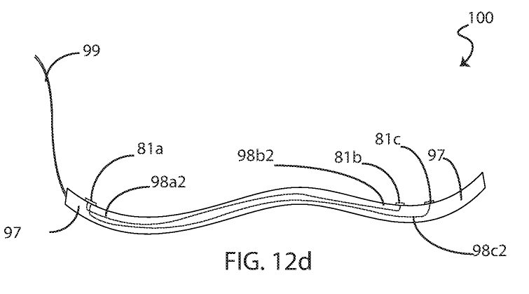

FIGS. 12a-b are perspective views of a shoe support member of a proprioceptive enhancement device mounted on a shoe. FIGS. 12a-b show a support member 50 which firmly but reversibly couples to the outside of most shoes, including shoes of different sizes, shapes, and styles. Support member 50 is conductive in some embodiments and nonconductive in some embodiments. In some embodiments wherein support member 50 is nonconductive, grounding members 52 are coupled to support member 50, as shown in FIG. 12b. A lead 51 coupled between shoe support member 50 and grounding member 52 is present in some embodiment, wherein lead 51 is fixedly or removably coupled to either support member 40 or grounding member 52. Lead(s) 51 comprise different lengths, and, if detachable, the different lengths may be used interchangeably with shoe support member 50 or grounding member 52 to accommodate different configurations of shoes and socks. An attachment for lead 51 (not shown) may comprise a friction-fit mechanism, a clip, a plug, a magnetic member, or other suitable means of reversibly coupling the lead 51 from shoe support member 50 or grounding member 52.

Lead 51, in some embodiments, is fixedly coupled to cutaneous electrode 81 that comprises an adhesive surface contacting skin of the user. Conducting electrode 81, in some embodiments, is non-adhesive and configured to be secured by placing it under the user's sock in contact with the skin. Cutaneous electrode 81 and the distal end of lead 51 may be coupled by a friction-fit type attachment means, if removable.

Shoe support member 50 and grounding member 52 are formed, in some embodiments, as relatively thin and unobtrusive because shoe support member 50 simply holds grounding member(s) 52 in position, wherein grounding member 52 contacts the earth or grounded standing surface to conduct an electrical current. The configuration of device 100 described above provides the user with a lightweight, compressible device that is easily portable, easily attached to most shoes, and which can be quickly coupled to any type of shoe at a time convenient to the user.

In use, shoe support member 50 is stretched over a shoe and cutaneous electrode 81 is positioned in contact with the skin at a preferred location on the foot, ankle or other convenient body region. Alternatively, instead of directly attaching cutaneous electrode 81 to a skin surface, cutaneous electrode 81 may be placed inside a sock (or shoe, if no sock is worn) to establish conductive contact with the user. Use of proprioceptive enhancing device 100 in pairs, wherein one device 100 is electrically coupled to each foot, grounds both feet of the user, recreating the natural condition wherein humans evolved walking barefoot outdoors.

Moreover, the embodiment shown in FIG. 12a-b may be alternatively formed wherein grounding member 52 is electrically coupled with a specific cutaneous region of the foot to more completely mirror the barefoot experience of our ancestry over hundreds of thousands of years. Natural ambulation consists of the heel striking the ground or walking surface first, quickly followed by a rolling motion of the plantar aspect of the foot which results in the heel being lifted from contact from the surface and weight and contact being transferred to the forefoot and then to the toes. This motion causes the heel to be the initial grounding body part, followed by the forefoot and then the toes. To recreate this natural chain of grounding events, some embodiments of device 100 provide electrical conductivity to the body through only that body part which would be touching the ground or ambulating surface if the subject were barefoot or was wearing conductive footwear. Such embodiments not only the benefits of grounding, but also provides the natural physiologic sequence of being grounded to the earth which may affect proprioception, balance, muscular coordination, and weight transfer and feedback amongst other important functions.

FIG. 12c is a plantar view of a shoe support member of a proprioceptive enhancement device. FIG. 12d is a cross-sectional view of an insole of a proprioception enhancement device. FIG. 12c shows shoe support member 50 that is easily detachable from the shoe or foot. As shown by FIG. 12d, an insole 97 comprises a plurality of individual wires 98. Insole 97 is formed similar to a commercially available insole, with the addition of one or more cutaneous electrodes 81 coupled to one or more corresponding wires 98 present within the substance of the insole. Each wire 98 is also coupled to a common terminus 99. Common terminus 99, in some embodiments, is an electrical harness-type connector comprising connections for a plurality of electrical circuits while electrically insulating each connection, such as a standard wiring harness of appropriate size for mounting on a shoe, as shown in FIG. 12a and FIG. 12c.

Some embodiments, including the embodiment shown in FIG. 12c and FIG. 12d, comprise a plurality of grounding members 52 configured such that grounding member 52a is coupled to a region of insole 97 contacting plantar skin of a heel of the user at a cutaneous electrode 81a. Grounding member 52b of the forefoot of shoe support member 50 is coupled to cutaneous electrode 81b contacting plantar skin of the user's forefoot area, and grounding member 52c coupled to shoe support member 50 is coupled to cutaneous electrode 81c contacting skin in the user's toe area. This may be accomplished by connecting the grounding members 52a, 52b and 52c with individual wires or other conductive means (not shown) and coursing them through or adjacent to elements of shoe support member 50 toward a common terminus 99, wherein corresponding individual wires 98a2, 98b2, and 98c2 of the insole terminate. Each individual wire 98 is electrically insulated from the other wires 98 so that any electrical activity is conducted from one specific area of shoe support member 50 only to the corresponding cutaneous electrode 81 coupled to insole 97. Although three areas of connectivity between the shoe support member 50 and specific areas of the skin of the foot are shown, between one and fifty different areas of connectivity may be utilized, although preferably between two and ten areas of connectivity are utilized.

The embodiments shown in FIGS. 12a-c, and some other embodiments, comprises shoe support member 50 formed from conductive materials but with insulating sections between regions contacting areas of the foot—the heel, the forefoot, and the toe areas, for example—so that contact with the earth of one area of shoe support member 50 will ground only that corresponding anatomical region of the foot cutaneous electrode 81, or a plurality of cutaneous electrodes 81. In some embodiments, shoe support member 50 is formed from strap of nonconductive material, such as a plastic or a synthetic rubber, for example. A conductive substance, which may be a wire-like or flat structure, or a conductive shapeable or malleable material is electrically coupled to grounding member 52 of shoe support member 50 at common terminus 99 to effect conductivity from grounding member 52 mounted at a specific area of the shoe support member 50 to a corresponding specific area of the skin of the foot via cutaneous electrode 81. The conductive substance, structure or material applied to the shoe strap may be connected to another means which may comprise a different substance, structure, or material to further conduct the electrical charge from the shoe strap to the inner aspect of the shoe and/or to the specific area of the foot.

Intermediate conductive means between grounding member 52 of shoe support member 50 and cutaneous electrode 81 applied to skin of the specific targeted area on the sole of the foot may utilize conductive shoe liners, insole liners, insoles or conductive elements coupled to insoles to provide conductivity. FIG. 12d shows insole 97 that are comprised by some embodiments of proprioceptive enhancement device 100. The insulated conductive wires 98a2, 98b2, and 98c2 are coupled to common terminus 99 which, in some embodiments, comprises a detachable member 99a that allows connectivity to the components of shoe support member 50 shown in FIG. 12d. Shoe support member 50, therefore, may be conveniently and easily used on different pairs of shoes which may be fitted with elements of device 100 shown in FIG. 12d without changing or modifying elements within the shoe.

Because most socks are conductive when the foot perspires a bit, the above configurations will provide means that physiologically mimic and recreate the grounding effects of walking barefoot by sequentially grounding the foot while walking with conductive footwear which may enhance balance, proprioception, muscle coordination, athletic prowess, and prevent imbalance, amongst other functions. Combining these elements with stimulation members 24 that may have been previously described herein, enhancements to human performance may even be accentuated.

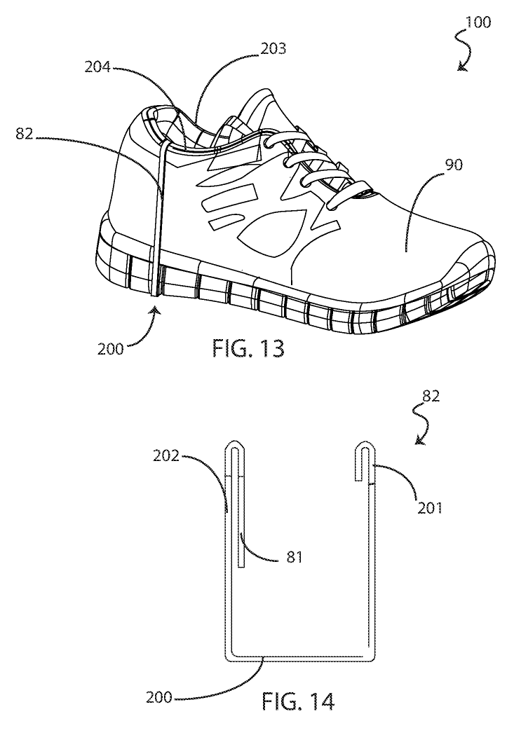

FIG. 13 is a perspective view of an alternative embodiment of a conductive member of a proprioceptive enhancement device mounted on a shoe. FIG. 13 shows an embodiment of device 100 that may be used with a conductive insole, such as insole 97 described herein. FIG. 14 is a front-view of an alternative embodiment of a conductive member of a proprioceptive enhancement device.

Conductive member 82, in the embodiment shown in FIG. 13 and FIG. 14, and in some other embodiments, comprises an external shoe strap 200 that extends between the a medial upper quarter edge 203 and a lateral upper quarter edge 204 upper quarter edges of shoe 90 (the borders of the large aperture into which one inserts the foot into shoe 90) by coursing under the sole of the shoe. Conductive member 82, in some embodiments, is constructed of an elastic conductive substance and is secured to both the medial and lateral upper quarter edges of the shoe by a slight tension on the elastomeric material. An attachment member 201, in some embodiments, is a small U-shaped component formed from a different material than the shoe strap. A conductive lead 202 of conductive member 82 is configured to be directed to the inside of the shoe to make electroconductive contact with one or more of cutaneous electrode 81, insole 97, a conductive insole, an insole with conductive components or filaments, an electrode, the user's skin, a sock-like garment, or other conductive component wherein conductive member 82 is electrically coupled to the skin of the user, generally at the foot and ankle region. In some embodiments, conductive lead 202 contact the user's skin outside of shoe 90, or other footwear.

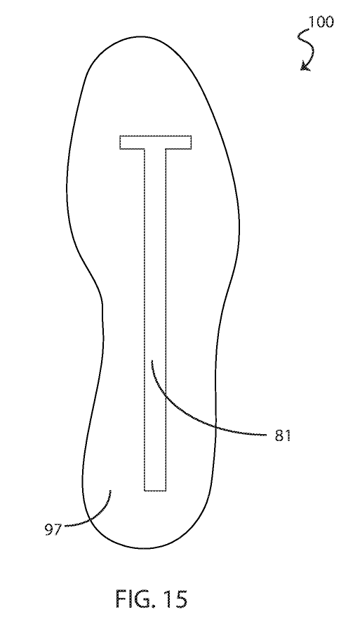

FIG. 15 is a top-view of an insole of a proprioceptive enhancement device. FIG. 15 shows insole 97 having cutaneous electrode 81 on an upper surface which contacts the plantar region of a user's foot when worn inside a user's footwear. In some embodiments, cutaneous electrode 81 comprises an adhesive surface that adheres to a standard non-conductive insole which may be provided with the footwear, such as a conductive tape or a tape like structure with conductive wires or components incorporated within or bonded to the tape like structure. In some embodiments, therefore, cutaneous electrode 81 comprises a conductive tape which is applied to the top of a conventional, non-conductive insole and secured in place by an adhesive substance. Conductive member 82, in some embodiments, also comprises conductive tape or an alternative conductive material, that electrically couples to the lead 51, conductive member 82, or grounding element 83 in some of the embodiments described herein, and some other embodiments.

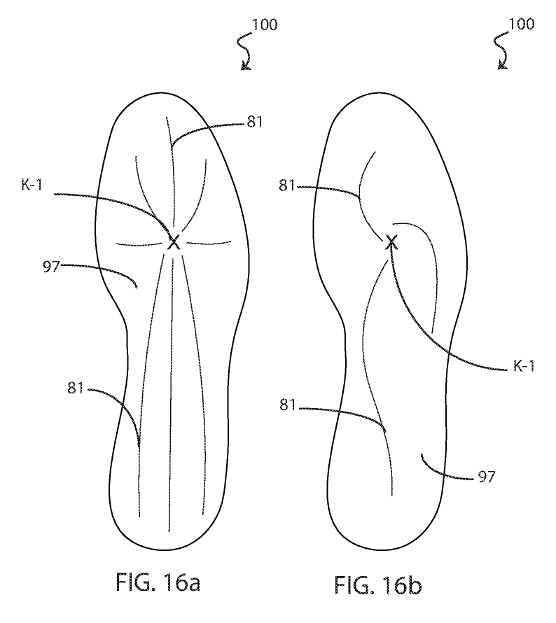

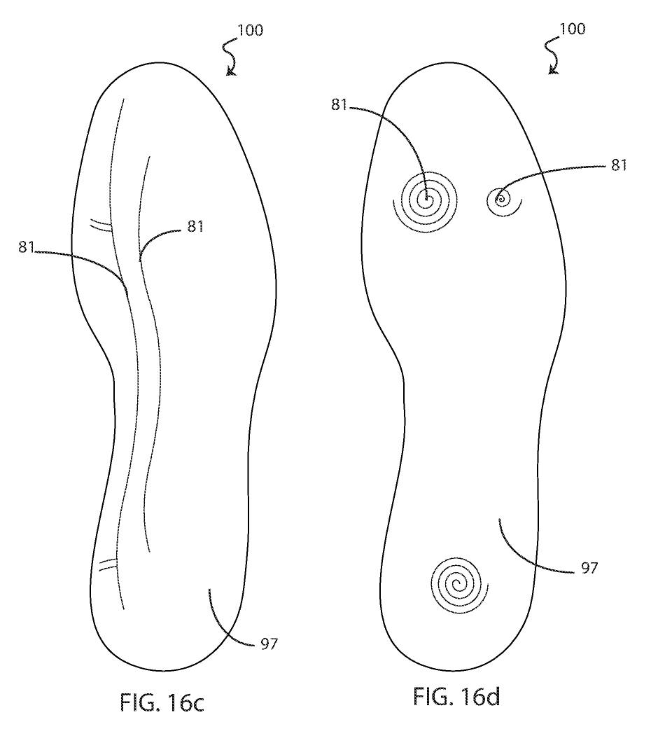

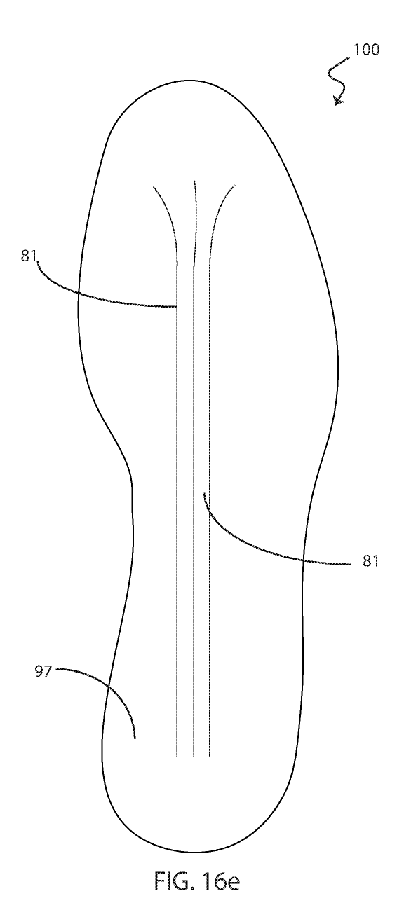

FIGS. 16a-e are top views of several alternative embodiments of a conductive insole of a proprioceptive enhancement device. FIGS. 16a-e show cutaneous electrode 81, in different embodiments, comprising patterns of distribution of conductive materials or filaments radiating out from an acupuncture meridian, such as a kidney, or “K-1” meridian. The K-1 or Kidney 1 acupuncture meridian area 100 is the convergence point of conductive filaments or materials which extend to other parts of the sole of the foot, including the toes. The toes are sensitive structures important in balance. A component of cutaneous electrode 81, in some embodiments, is posteriorly to electrically coupled with common terminus 99 discussed herein above, wherein a user of device 100 is electrically grounded to the earth, directly or through a grounded standing surface, through grounding member 52 coupled to cutaneous electrode 81 via common terminus 99, at an acupuncture meridian.

As described herein above, in some embodiments, garment 20 comprises any combination of stimulation member 24, unpleasant stimulation member 66, stimulation member 124, unpleasant stimulation member, and cutaneous electrode 81 electrically coupled to grounding member 52. In fact, any or all of the physical stimulating means to enhance balance described earlier may be combined with the conductive grounding means to enhance balance. Further, in some embodiments, the conductive means additional becomes a stimulating means, as wherein the conductive means are filaments or wires that course through the material of the sock or the inner sole, for example. Also, as shown by FIGS. 16a-e, cutaneous electrode 81 comprising conductive filaments is positioned such that cutaneous electrode 81 contacts the plantar skin at specific regions most desirable for weight-bearing, and, additionally, provides grounding to these areas. The filaments may also be of a size or configuration that is physically stimulating as has been previously discussed. Placement of these filaments in areas of desired weight bearing will urge and remind the user to position the feet and the weight on the feet for optimal balance, or in the case of sports or other activities, in the best position to perform the desired activity. In the case of golf, it is usually advantageous to place one's weight on the inner aspects of both feet. Cutaneous electrode 81 comprising conductive filaments, therefore, is configured for skin contact medially, where a user's weight should be concentrated. As shown by FIG. 16b, cutaneous electrode 81 is positioned on insole 97 in the areas of the foot for optimal balance in age-related balance disorders. The cutaneous electrodes 81 shown in the embodiments of FIGS. 16a-c, and in some other embodiments, are electrically coupled to common terminus 99 as previously discussed to complete the grounding connection.

In some embodiments, the configuration of garment 20 is optimized for walking with any combination of stimulation member 24, stimulation member 124, unpleasant stimulation member 66, unpleasant stimulation member 144, and cutaneous electrode 81, contacting any location of the user's foot, including a location extending from the center heel pad region to the K-1 meridian region. This configuration would remind the user to place the heel pad on the walking surface first and then followed by the center of the forefoot and may prevent weight from being placed laterally on the foot, which predisposes one to falls. If combined with the sequential grounding means of FIGS. 16c-d, which may enhance sensitivity to these stimuli by providing the natural conductivity sequence of events, not only the prevention of imbalance and falls may be enhanced, but various athletic performances may be enhanced.

Some embodiments of device 100 comprise shoe 90 which is conductive, but provides the resiliency, comfort, durability and dryness of a composite or non-natural materials. This is accomplished wherein grounding member 52 is a conductive substance or structure formed as a unitary member with a sole or other parts of shoe 90. Electrical coupling between the plantar skin of a user of device 100 and the earth is established wherein the sock, insole, and shoe are all conductive. In the instance of moisture being present, a non-conductive sock may become conductive if there is enough perspiration. This is uncomfortable however, and consistent conductivity may not be achievable if the socks are not conductive or have conductive means within them. Many different configurations of device 100 may be utilized in several different embodiments, including device 100 comprising a sock or insole with conductive means which contact a sole-piercing grounding member 52 or other conductive members wherever they may be located, and either transmits the grounding to the sole of the foot at or near the Kidney 1 acupuncture meridian or directly to the skin of the foot near the sole piercing or other conductive member. This may be accomplished wherein cutaneous electrode 81 comprises a conductive thread or filaments penetrating from grounding member 52 through a standard sock, electrically coupling the plantar skin at the Kidney 1 acupuncture meridian. Patterns of cutaneous electrode 81 described herein, or alternative patterns, are utilized in some embodiments.

Some embodiments of device 100 comprise a sock wherein fibers of cutaneous electrode 81 emanate from the K-1 area and is directed towards at least the medial aspect of the foot where it may contact grounding member 52 directly or indirectly through electrical coupling with conductive member 82. A shoe support member couples to shoe 90, either releasably or fixedly, or, in some embodiments, does not attach to shoe 90, but contacts the ground or grounded standing surface directly from the sock. In either case, cutaneous electrode 81 of the “grounding sock” and grounding member 52 will effectively ground the user without a special shoe, wherein the user may use their current shoes, boots and sandals. The grounding sock may be constructed of conductive fibers throughout the weave of the material or with special conductive filaments inserted in a nonconductive weave material.

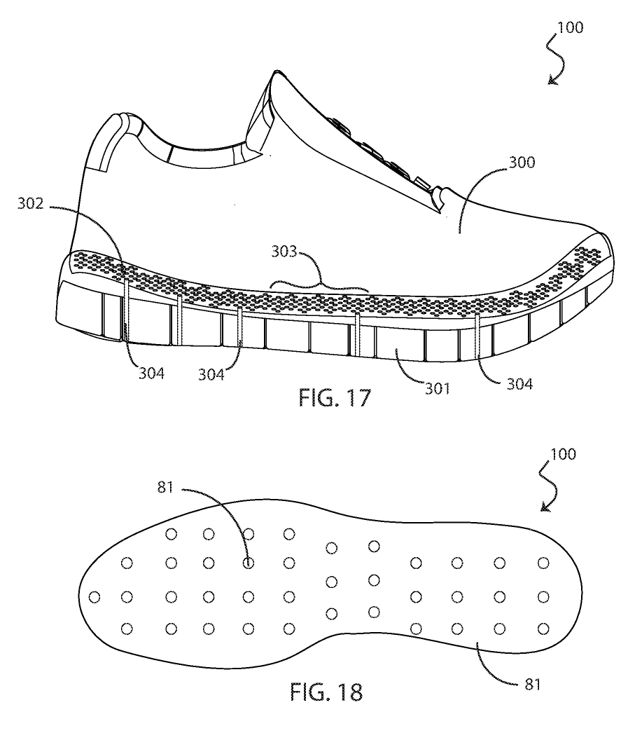

FIG. 17 is a medial cross-sectional view of a sole mounted on a shoe of a proprioceptive enhancement device. FIG. 17 shows a partial-cutaway view of proprioception enhancing device 100 comprising a shoe 300 showing a sole 301 comprising an inner surface 302 that forms a plurality of internal terminations 303. Internal terminations 303 function as cutaneous electrodes and, in some embodiments, are formed from electrically conductive strips, wires, polymers, filaments or columns of a conductive substance 304, molded or injected conductive substances or the like. FIG. 17 also shows a connecting element 304 traversing sole 301 to the exterior. In some embodiments, connecting element 304 is columns or channels of formed from any of many suitable conductive materials. They may be inserts. The illustration is just for one configuration of multiple conductive elements that connect the wearer with the ground progressively by utilizing multiple means that cause the interior of the shoe sole to reflect the electric potential of the earth when walking by being grounded in the heel region, then the midfoot, the forefoot and finally the toes. The heel area and midfoot may become ungrounded as the step progresses, to be repeated again. Humans are used to being grounded in this manner Sleeping on a grounded sheet does not replicate the human experience. Humans are programmed in our evolved DNA to function optimally while 1) being grounded to the earth, 2) grounded to the earth during the right times of day, and 3) with progressive grounding of the feet when walking. The designs herein also increase the sensitivity of balance and proprioception when standing with grounded footwear as described. This device replicates the evolutionary human experience by progressively grounding the sole of the foot. A device in which the connecting means 104 extend to the tip of a golf spike or cleat on a soccer or football shoe and into the earth will provide loco-regional or focal conductivity with the earth during sporting events and is a part of this invention. Sports performance may be enhanced by simply grounding the individual, and potentially even further by replicating the grounding pattern of our evolutionary history.

FIG. 18 is a top-view of an alternative embodiment of an insole of a proprioceptive enhancement device.

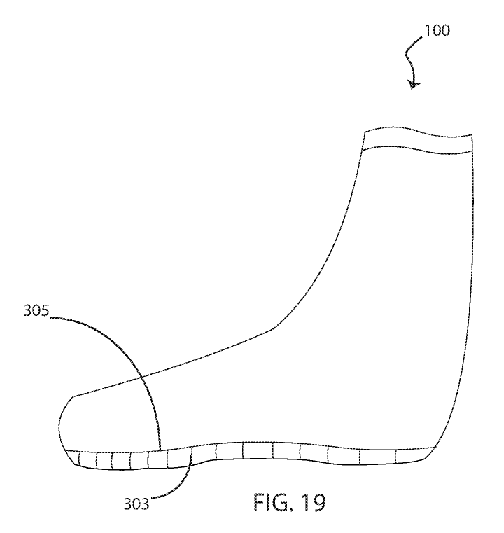

FIG. 19 is a side view of a conductive sock of a proprioceptive enhancement device. FIG. 19 shows an inner sole 305 having a connecting element 106. In this and some other embodiments, sole conductive member 303 creates an electrical connection between the ground to a specific area of plantar skin that would contact the ground directly if the subject were barefoot and not wearing a shoe or other footwear. Thin, relatively flat conductive members (not shown), or the like, are used in some embodiments between inner sole 305 and inner surface of sole 302 of shoe 300 in this and some other embodiments to insure that sole conductive members 303 and inner sole conductive members 303 remain in conductive electrical contact if there is any movement of the inner sole 305 with respect to shoe 300. This and some other embodiments also permit a variety of configurations of conductive inner soles to function in a variety of shoe configurations and allow interchangeability of inner soles and shoes. Alternatively, the entire inner sole may be constructed of a conductive material which may obviate the need for the thin, relatively flat conductive member.

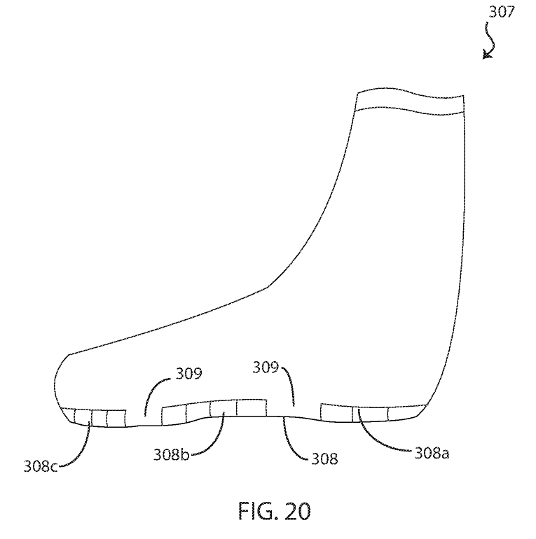

FIG. 20 is a side view of an alternative embodiment of a conductive sock of a proprioceptive enhancement device. FIG. 20 shows a sock 307 and a sole 308 comprising separate areas of conductivity 308a, 308b, and 308c which focally ground the heel, forefoot, and toe areas, respectively, of the foot. Sock 307 and sole 308, in some embodiments, are formed from and comprise a conductive material, and a remainder 309 is formed from a nonconductive material. This and some other embodiments closely mimic the actual grounding experienced by our forbearers over the ages than just generic grounding as some of the other means and methods described herein. Grounding the whole foot or only portions of it may not be as advantageous as recreating the environment that persisted for ages. Grounding the whole foot and ankle with a conductive sock that covers the whole foot and ankle area does not mimic the evolutionary history of grounding as much as providing a sock that is only conductive in the sole portion.

The components defining any proprioceptive enhancement device may be formed of any of many different types of materials or combinations thereof that can readily be formed into shaped objects provided that the components selected are consistent with the intended operation of a proprioceptive enhancement device. For example, the components may be formed of: rubbers (synthetic and/or natural) and/or other like materials; glasses (such as fiberglass) carbon-fiber, aramid-fiber, any combination thereof, and/or other like materials; polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; composites and/or other like materials; metals, such as zinc, magnesium, titanium, copper, iron, steel, carbon steel, alloy steel, tool steel, stainless steel, aluminum, any combination thereof, and/or other like materials; alloys, such as aluminum alloy, titanium alloy, magnesium alloy, copper alloy, any combination thereof, and/or other like materials; any other suitable material; and/or any combination thereof.

Furthermore, the components defining any proprioceptive enhancement device may be purchased pre-manufactured or manufactured separately and then assembled together. However, any or all of the components may be manufactured simultaneously and integrally joined with one another. Manufacture of these components separately or simultaneously may involve extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled with one another in any manner, such as with adhesive, a weld, a fastener (e.g. a bolt, a nut, a screw, a nail, a rivet, a pin, and/or the like), wiring, any combination thereof, and/or the like for example, depending on, among other considerations, the particular material forming the components. Other possible steps might include sand blasting, polishing, powder coating, zinc plating, anodizing, hard anodizing, and/or painting the components for example.

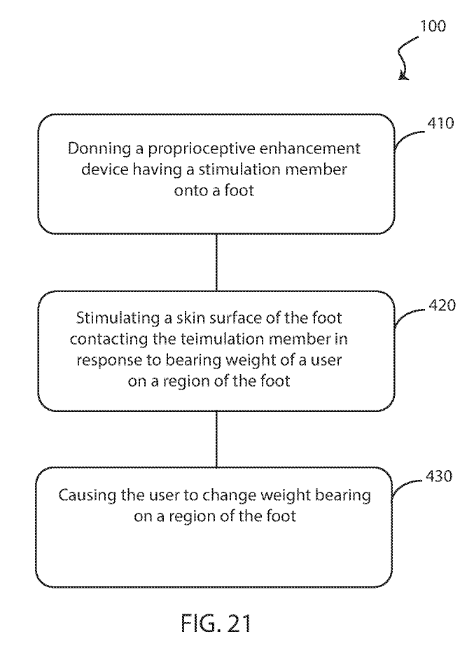

FIG. 21 is a flowchart showing a method 400 of treating a foot wound utilizing a proprioceptive enhancement device. Method 400 comprises an earthing step 410, a stimulating step 420, and a causing step 430.

Earthling step 410 is performed by donning a footwear item having a stimulating member, a grounding element, a conductive member electrically coupled to the grounding element, and a cutaneous electrode, wherein the cutaneous electrode contacts a user's skin at the foot and ankle region of the user.

Stimulating step 420 is performed when the user dons a footwear garment of the proprioceptive enhancement device, wherein a stimulation member contacts the user's plantar skin and creates mechanical stimulation, electrical stimulation, or a combination of electrical and mechanical stimulation, in some embodiments of method 400.

Causing step 430 is performed following stimulating step 410, wherein mechanical, electrical, or a combined electromechanical stimulation causes increased blood flow to the foot wound. Increased blood flow tends to increase local tissue oxygen tension, and additionally delivers glucose and trophic bioactive molecular species widely known in the medical arts to support and enhance wound healing. Increased blood flow additionally delivers increased number of immune-reactive lymphocytes and serum antibodies to the wound to prevent and treat infection which would otherwise prevent or delay wound healing.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

What is claimed is:

1. A proprioceptive enhancement device comprising:
a foot garment having
a cutaneous electrode and
a pressure transducer; and
a current generator electrically coupled to the cutaneous electrode, wherein the current generator delivers a current to the cutaneous electrode in response to a signal generated by the pressure transducer.

2. The proprioceptive enhancement device of claim 1, further comprising
a microprocessor communicatively coupled to the pressure transducer and the current generator; and
an algorithm resident on microprocessor, wherein the algorithm directs the microprocessor to signal the current generator to deliver the current to the cutaneous electrode in response to the signal generated by the pressure transducer.

3. A proprioceptive enhancement device comprising:
a foot garment having an array, wherein the array comprises a plurality of stimulation members coupled to the foot garment;
a conductive member coupled to the foot garment; and
a shoe having a grounding element, wherein the grounding element is electrically coupled to the conductive member.

4. The proprioceptive enhancement device of claim 3, wherein the plurality of stimulation members are detachably coupled to the foot garment.

5. The proprioceptive enhancement device of claim 3, wherein the plurality of stimulation members comprise a cutaneous electrode.

6. The proprioceptive enhancement device of claim 3, wherein the plurality of stimulation member comprise a protrusion.

7. The proprioceptive enhancement device if claim 3, wherein the plurality of stimulation members comprise a cutaneous electrode and a protrusion.

8. The proprioceptive enhancement device of claim 3, wherein a position of the array on the foot garment is adjustable.

9. The proprioceptive enhancement device of claim 3, wherein a shape of the array is adjustable.

10. The proprioceptive enhancement device of claim 3, wherein the array is positioned on the foot garment corresponding with a position of an acupuncture meridian on a foot of a user.

11. The proprioceptive enhancement device of claim 10, wherein the acupuncture meridian is a Kidney Meridian.

12. The proprioceptive enhancement device of claim 3, wherein the array is positioned proximate to an ankle joint.

13. The proprioceptive enhancement device of claim 3, further comprising an actuator operatively coupled to the grounding element, wherein the ground element is biased against a ground surface by the actuator.

14. The proprioceptive enhancement device of claim 3, further comprising a shoe support member coupled to the ground element and the connector.

15. A method of treating a foot wound comprising steps:
donning a proprioceptive enhancement device having a stimulation member onto a foot;
stimulating a skin surface of the foot contacting the stimulation member in response to bearing weight of a user on a region of the foot; and
causing the user to change weight bearing on a region of the foot.

16. The method of claim 14, wherein the stimulating step comprises:
generating a signal from a pressure transducer coupled to the proprioceptive enhancement device in response to bearing weight of a user on a region of the foot; and
activating a current source to energize a cutaneous electrode coupled to the proprioceptive enhancement device.

17. The method of claim 16, further comprising a step electrically grounding the foot to the earth by a ground element electrically coupled to the foot by the stimulation member.
